(19) United States
(12) Patent Application Publication
(10) Pub. No.: US 2014/0163048 A1
(43) Pub. Date: Jun. 12, 2014

(54) COMPOSITIONS WITH INCREASED STABILITY FOR INHIBITING TRANSIENT RECEPTOR POTENTIAL ION CHANNEL TRPA1

(71) Applicant: Cubist Pharmaceuticals, Inc., Lexington, MA (US)

(72) Inventors: Nicholas Barker, Lexington, MA (US); Jian-Qiao Gu, Lexington, MA (US); Gaauri NAIK, Lexington, MA (US); Jan-Ji LAI, Lexington, MA (US); Stephen MACHATHA, Lexington, MA (US); You Seok Hwang, Lexington, MA (US); Pradip M. Pathare, Lexington, MA (US)

(73) Assignee: Cubist Pharmaceuticals, Inc., Lexington, MA (US)

(21) Appl. No.: 13/963,011

(22) Filed: Aug. 9, 2013

Related U.S. Application Data
(60) Provisional application No. 61/681,506, filed on Aug. 9, 2012.
(60) Provisional application No. 61/798,156, filed on Mar. 15, 2013.

(51) Int. Cl. C07D 473/08

(52) U.S. Cl. CPC C07D 473/08
USPC 514/263.21; 544/270

(57) ABSTRACT

This disclosure describes solid forms of the compound of Formula (I) and pharmaceutical compositions for inhibiting the TRPA1 ion channel and/or medical conditions related to TRPA1, such as pain.

### RELATED APPLICATION

The present application claims priority to U.S. Provisional Application No. 61/681,506, filed Aug. 9, 2012, and claims priority to U.S. Provisional Application No. 61/798,156, filed Mar. 15, 2013, both of which are incorporated herein by reference in their entireties.

### TECHNICAL FIELD

The present disclosure relates to solid forms of a compound for treating pain, for example by inhibiting the Transient Receptor Potential A1 ion channel (TRPA1). Pharmaceutical compositions comprising the compound are also provided herein.

### BACKGROUND

Delivering an active pharmaceutical ingredient (API) to a patient generally involves more than just identifying a molecule and its use. The API must be formulated for delivery to a patient, and evaluated by regulatory agencies. A regulatory agency can evaluate an API formulation for, among other properties, delivery properties, stability, and manufacturing controls. An important factor in determining the properties of a particular formulation is the form of the API. For example, APIs can be formulated as amorphous forms, crystalline forms, polymorphs, hydrates, or solvates of a compound. The particular form with the most favorable properties can be different for each API. Thus, form diversity is a consideration in API formulation because each different polymorph, solvate, hydrate or amorphous form can have different properties such as stability and solubility.

Some forms of an API can be formulated into safe and effective drug products, while other API solid forms can lack the required properties to be safe and/or effective. Even if a particular API can exist in more than one safe and/or effective formulation, different properties of the various solid forms of a given API can affect other drug product properties, such as the manufacturing process, shelf stability, route of administration, and bioavailability. For example, identifying safe and effective solid forms of an API can improve or modulate stability of a drug product or can increase product shelf-life thereby improving product distribution possibilities. In addition, one solid form of an API may have greater oral bioavailability than another form.

There is a need to identify API solid forms with desirable levels of oral bioavailability, for example to provide for a lower API dose to be administered to the patient in a drug product. Different solid forms of the same API may have drastically different oral bioavailability. Thus, identifying solid forms with high oral bioavailability increases the opportunity to identify the ideal form for approval and eventual patient treatment.

TRPA1 is a non-selective cation channel related to pain sensation in humans. TRPA1 is found in sensory neurons and functions as a signal transduction receptor linking inflammation to pain. TRPA1 underlies pain related to nerve damage, cold allodynia, and inflammatory pain. TRPA1 inhibitor compounds can be used to treat pain. Compounds that inhibit the TRPA1 ion channel can be useful, for example, in treating conditions ameliorated, eliminated or prevented by inhibition of the TRPA1 ion channel (e.g., medical conditions causing pain). Applicants have discovered the compound of Formula (I), and salts thereof (e.g., pharmaceutically acceptable salts), can form novel solid forms possessing distinct physical properties and distinct crystal structures. This discovery increases the opportunity for the identification of an improved formulation with properties favorable to the manufacturing process, shelf stability, ease of administration, bioavailability and ultimate FDA approval.

### SUMMARY

The compound of Formula (I) is an antagonist of the human TRPA1 channel.

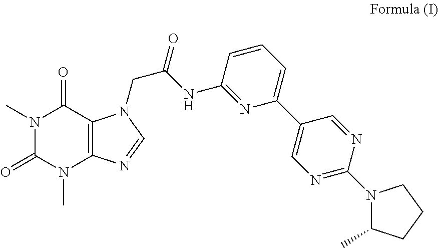 [SMILES: C[C@H]1CCCN1c1ncc(-c2cccc(NC(=O)Cn3cnc4c3c(=O)n(C)c(=O)n4C)n2)cn1]

The free base of the compound of Formula (I) can be synthesized according to the synthesis of FIG. 1A, as described in Example 1A. The free base compound of Formula (I) can also be used as a starting material for making the mesylate salt of the compound of Formula (I) (FIG. 13). The free base compound of Formula (I) used as a starting material for making solid crystalline forms of the compound of Formula (I). One preferred solid crystalline form of the compound of Formula (I) is Form 1 (Example 2) and is identified by its characteristic X-ray powder diffraction (XRPD) peaks (Example 3). The solid Form 1 of the compound of Formula (I) has high solubility (Example 4) and stability (Example 5). Form 1 of the compound of Formula (I) has unexpectedly high oral exposure in dog (Example 6) making it potentially useful as a drug product.

Another preferred solid crystal from of the compound of Formula (I) is the novel crystal Form 4 which can be made according to Example 7. The solid Form 4 of the compound of Formula (I) can be identified by a characteristic XRPD pattern (Example 8).

Thirteen other novel crystal forms of the compound of Formula (I) can also be made and may be useful as drug products (Example 9). These novel crystal forms can also be identified by characteristic XRPD patterns (Table 6).

Solid crystal forms of the compound of Formula (I) can be made into pharmaceutical compositions that can be used to treat pain (Example 10).

The compound of Formula (I) is a highly selective in vitro inhibitor of TRPA1. For example, the compound of Formula (I) blocks inward currents through TRPA1 in rat, dog and human TRPA1 (Example 11). The antagonist effect of the compound of Formula (I) against human TRPA1 (hTRPA1) was measured in a whole cell patch configuration (Example 11). Furthermore, the compound of Formula (I) is highly selective for TRPA1 as compared with known TRP channels and voltage-gated ion channels (Example 11).

The compound of Formula (I) is also active in multiple in vivo rat models of pain, including pain induced by direct activation of the TRPA1 channel with formalin injection (Example 12b), cold allodynia following chronic Complete Freund's Adjuvant-induced inflammation (Example 12c), and a rodent surgical model involving the incision of the plantar surface of the hind paw (Example 12d).

Pharmaceutical compositions comprising the compound of Formula (I), and pharmaceutically acceptable salts and formulations thereof (e.g., pharmaceutical compositions including the compound of Formula (I) combined with a cyclodextrin), are useful in the treatment of pain, including inflammatory and post-operative pain. The compound of Formula (I), and pharmaceutically acceptable salts thereof, is also useful as a research tool, for example in assays including the modulation of the TRPA1 ion channel.

### BRIEF DESCRIPTION OF THE DRAWINGS

FIG. 1A is a reaction scheme to synthesize a compound of Formula (I), as described in Example 1A.

FIG. 1B is a reaction scheme to synthesize a deuterated compound (12), a deuterated analog of the compound of Formula (I), as described in Example 1B.

FIG. 2 is the unique XRPD pattern of the solid crystalline Form 1 of the compound of Formula (I).

FIG. 3 is the unique ¹H-NMR spectrum of the solid crystalline Form 1 of the compound of Formula (I).

FIGS. 4A-4D are a set of line graphs showing the increased solubility of the solid crystalline Form 1 of the compound of Formula (I) as compared to the free base of the compound of Formula (I) in SGF (simulated gastric fluid) and SGF spiked SIF (simulated intestinal fluid) experiments. FIG. 4A depicts the kinetic solubility of Form 1 of the compound of Formula (I) in simulated gastric fluid (SGF). FIG. 4B depicts the kinetic solubility of Form 1 of the compound of Formula (I) in SGF spiked simulated intestinal fluid (SIF). FIG. 4C depicts the kinetic solubility of the freebase of the compound of Formula (I) in SGF. FIG. 4D depicts the kinetic solubility of the freebase of the compound of Formula (I) in SGF spiked SIF.

FIG. 5 consists of two XRPD patterns, one measured before the solid crystalline Form 1 of the compound of Formula (I) underwent stability testing and one after storing a sample in ambient conditions (25° C./40% RH) for 7 days. FIG. 5 shows that the solid crystalline Form 1 of the compound of Formula (I) is stable under these conditions because there is no shift in the XRPD pattern.

FIG. 6 is a box plot representing the increased oral exposure of the solid crystalline Form 1 of the compound of Formula (I) as compared to other formulations of the compound of Formula (I) in dogs.

FIG. 7 is the unique XRPD pattern of the solid crystalline Form 4 of the compound of Formula (I).

FIG. 8 is a bar graph demonstrating the effect of administering a pharmaceutical composition comprising the compound of Formula (I) at different concentrations (3, 10, 30, and 50 mg/kg) to rodents prior to conducting a formalin injection as described in Example 12b. FIG. 8 shows the measured pain duration (as (n) seconds over a 2 minute observation period) in a rodent formalin injection pain model for various pharmaceutical compositions containing different amounts of the compound of Formula (I), a vehicle delivered intraperitoneally (i.p.), and the comparator compound of Formula (II).

FIG. 9 is a line graph demonstrating increased Paw Withdrawal Latency (PWL) scores observed after i.p. administration of pharmaceutical compositions with increasing concentrations of a compound of Formula (I) in the Complete Freund's Adjuvant (CFA) rodent model described in Example 12c. FIG. 9 shows the change in PWL score as a function of the concentration of a compound of Formula (I), as well as the PWL scores observed upon administration of the vehicle alone and a comparator pharmaceutical composition containing the comparator compound of Formula (II).

FIG. 10 is a line graph demonstrating reduction in guarding scores observed after i.p. administration of pharmaceutical compositions with various concentrations of the compound of Formula (I) in the rodent incisional pain model described in Example 12d. FIG. 10 shows the change in guarding score as a function of the administered concentration of the compound of Formula (I), as well as the guarding scores observed upon administration of the vehicle alone and comparator pharmaceutical compositions containing the comparator compound of Formula (III) or ketoprofen.

FIG. 11A is a bar graph of data for measurement of serum chemistry biomarkers of hepatotoxicity measured in female dogs orally dosed with a compound of Formula (I).

FIG. 11B is a bar graph of data for measurement of serum chemistry biomarkers of hepatotoxicity measured in male and female dogs orally dosed with a comparator compound of Formula (III).

FIG. 12 is a bar graph of data showing the effect on hepatotoxicity biomarkers in rat serum for administering a compound of Formula (I) or a comparator compound of Formula (III) in a 7-day i.p. repeat dose screening toxicity study at 50 mg/kg/day for 7 consecutive days.

FIG. 13 shows the synthesis of the mesylate salt of the compound of Formula (I).

FIG. 14 shows PK studies in dogs comparing the plasma concentration of the mesylate salt of the compound of Formula (I) to the hydrochloride salt of the compound of Formula (I).

### DETAILED DESCRIPTION

The compound of Formula (I) and pharmaceutically acceptable salts thereof, are useful for the inhibition of the TRPA1 ion channel in pharmaceutical compositions as well as research tools. The free base of the compound of Formula (I) can be synthesized and used as a starting material for making solid crystalline forms of the compound of Formula (I). The preferred solid crystalline form of the compound of Formula (I) is designated herein as “Form 1.” The solid Form 1 of the compound of Formula (I) is a novel crystal form which can be identified by a characteristic XRPD pattern and ¹H-NMR spectrum. The solid Form 1 of the compound of Formula (I) has high solubility and stability and has unexpectedly high oral exposure making it potentially useful as a drug product.

Another preferred solid crystal from of the compound of Formula (I) is the novel crystal Form 4. The solid Form 4 of the compound of Formula (I) can be identified by a characteristic XRPD pattern. Thirteen other novel crystal forms of the compound of Formula (I) can also be made and may be useful as drug products. These novel crystal forms can also be identified by characteristic XRPD patterns.

Solid crystal forms of the compound of Formula (I) can be made into pharmaceutical compositions that can be used to treat pain.

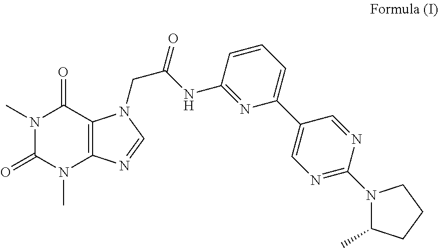 [SMILES: C[C@H]1CCCN1c1ncc(-c2cccc(NC(=O)Cn3cnc4c3c(=O)n(C)c(=O)n4C)n2)cn1]

### Synthesis of the Compound of Formula (I) Free Base

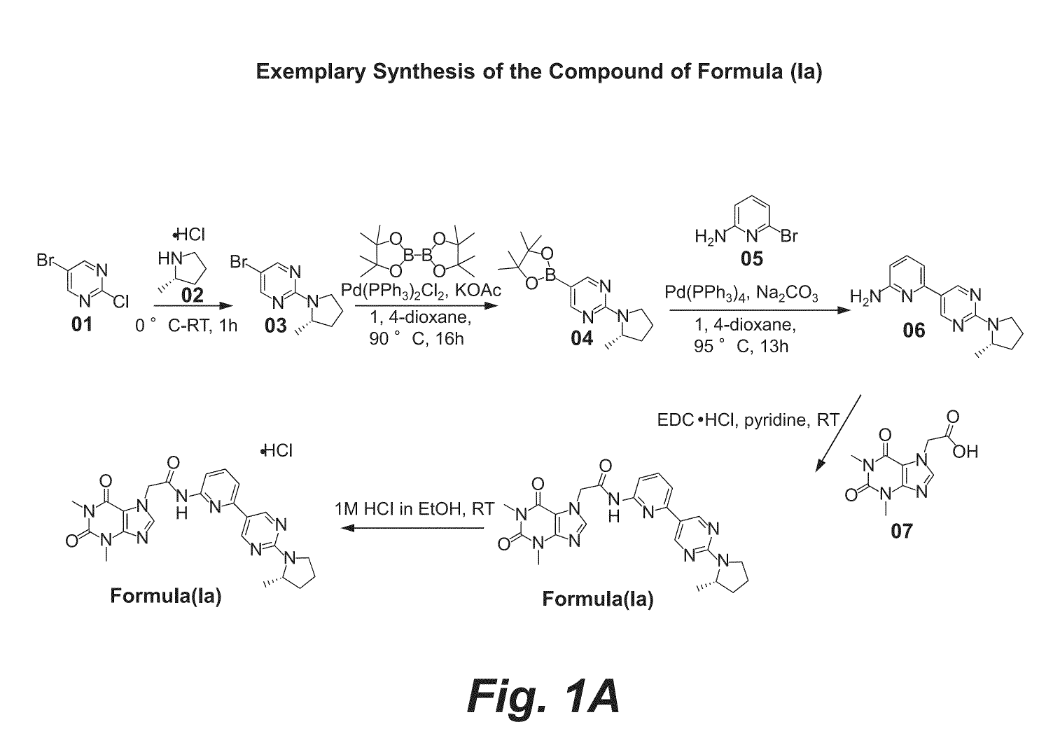

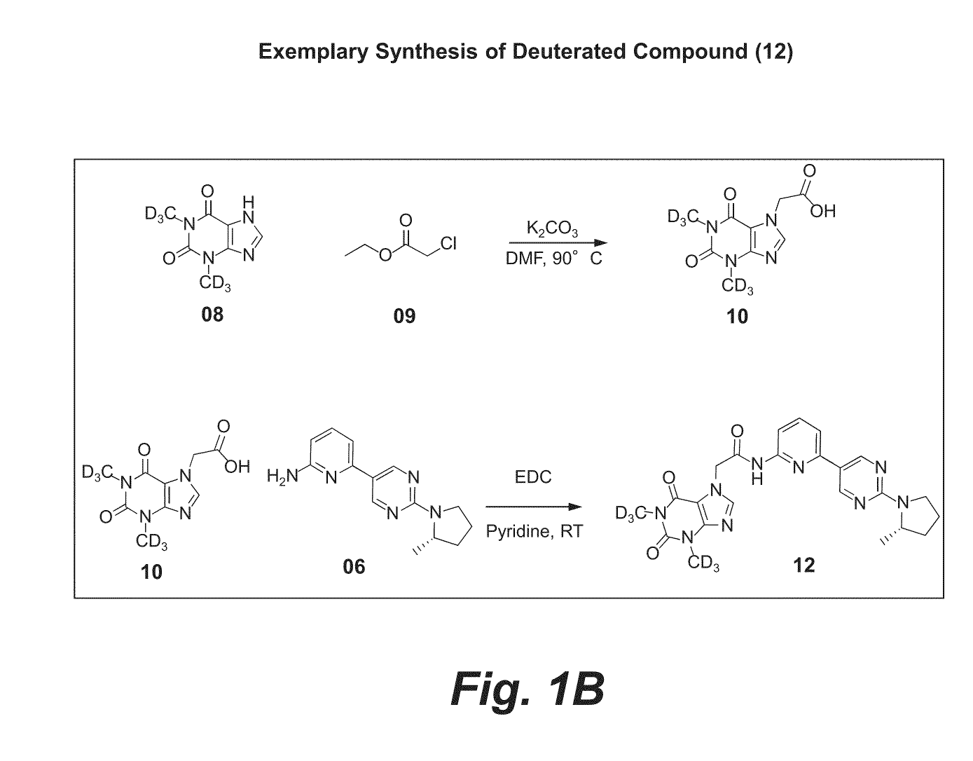

The compound of Formula (I) can be made by multi-step synthetic processes shown in FIG. 1A, as described in Example 1A. Briefly, referring to FIG. 1A, the compound of Formula (I) can be formed by: (1) reacting (S)-2-methylpyrrolidine with 5-bromo-2-chloropyrimidine to form the intermediate compound 03; (2) coupling the compound 03 intermediate with compound 05 (6-bromo-2-aminopyridine) by one or more reactions to form the intermediate compound 06; and (3) reacting compound 06 with compound 07 in a coupling reaction to form the compound of Formula (I). While coupling of the compound 03 intermediate with compound 05 can be performed via the intermediate compound 04, as shown in FIG. 1A and described in Example 1A, other synthetic schemes are also suitable for preparation of the compound of Formula (I). As described in Example 1A and FIG. 1A, the intermediate compound 06 can be formed by reacting compound 03 with bis(pinacolato)diboron and other materials to form the intermediate compound 04, followed by reaction of the intermediate compound 04 with 6-bromo-2-aminopyridine (compound 05) to obtain the intermediate compound 06. Each of the reaction steps can be formed with suitable reagents with reaction conditions suitable for obtaining the product(s) indicated in FIG. 1A.

Optionally, the process for synthesizing the compound of Formula (I) can further include steps for isolating the intermediate compounds 03 and compound 06 prior to performing subsequent reactions. In addition, the compound of Formula (I) can be optionally converted to a pharmaceutically acceptable salt. Solid forms of the compound of Formula (I) can be made and characterized according to Example 4.

### Formation of an Amorphous Form of the Mesylate Salt of the Compound of Formula (I)

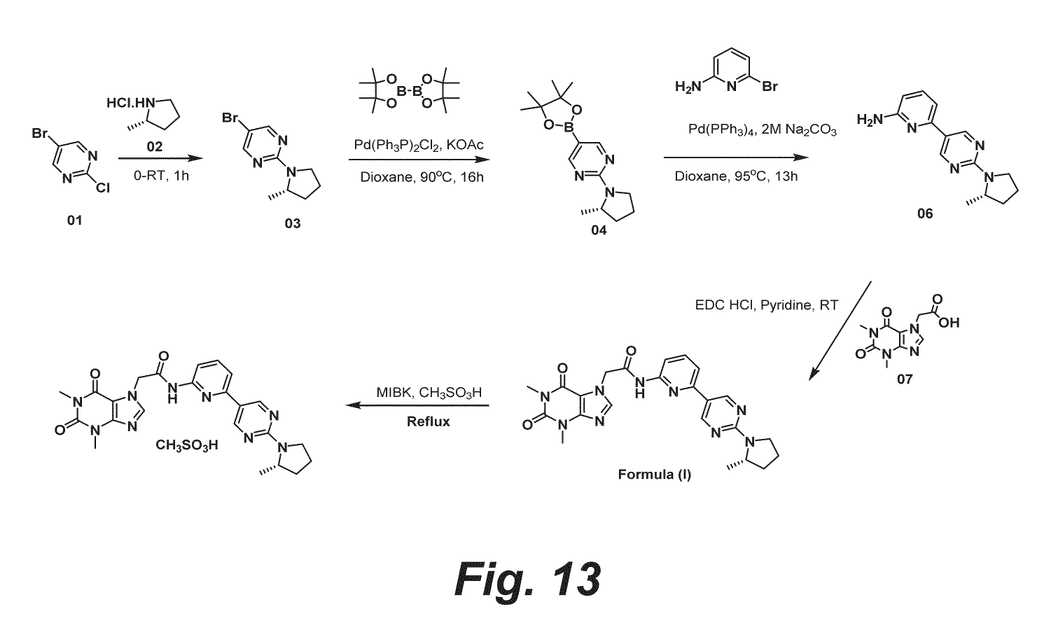

An amorphous form of the mesylate salt of the compound of Formula (I) can be formed by preparing the free base of the compound of Formula (I), which can be synthesized in accordance with the procedure described above, followed by reaction with methanesulfonic acid. This synthesis process is shown in FIG. 13.

### Synthesis of Solid Forms of the Compound of Formula (I)

Fifteen solid crystalline forms of the compound of Formula (I) were made using either amorphous compound of Formula (I), free base compound of Formula (I) or other crystalline forms of the compound of Formula (I) as starting materials. The preferred solid crystalline form of the compound of Formula (I) is Form 1. The solid Form 1 of the compound of Formula (I) has high solubility and stability and has unexpectedly high oral exposure making it particularly useful as a drug product. Another preferred solid crystalline form of the compound (I) is Form 4. The remaining thirteen solid crystalline forms of the compound of Formula (I) represent a diversity of forms some of which may possess qualities favorable to incorporation into an API.

The preferred solid forms of Formula (I) are pharmaceutically acceptable salts. The term, “pharmaceutically acceptable salts” of the compound of Formula (I), refers to salts prepared from pharmaceutically acceptable non-toxic acids including inorganic acids and organic acids. In general, pharmaceutically acceptable salts of Formula (I) can be prepared to improve stability or toxicological properties of the compound, increase or decrease solubility, wetability, improve pharmacokinetic performance of the compound (e.g., Cₘₐₓ or AUC measurements) or improve storage properties (e.g., to reduce hygroscopicity) of a pharmaceutical composition.

### Formation of the Solid Form 1 of the Compound of Formula (I)

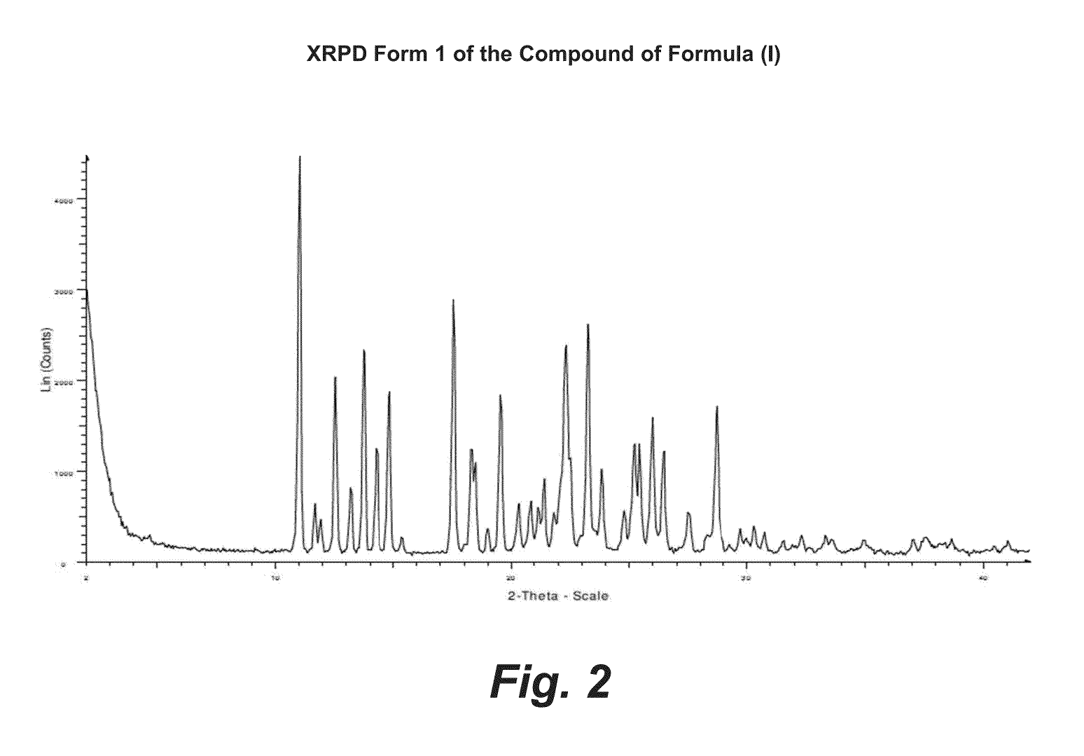

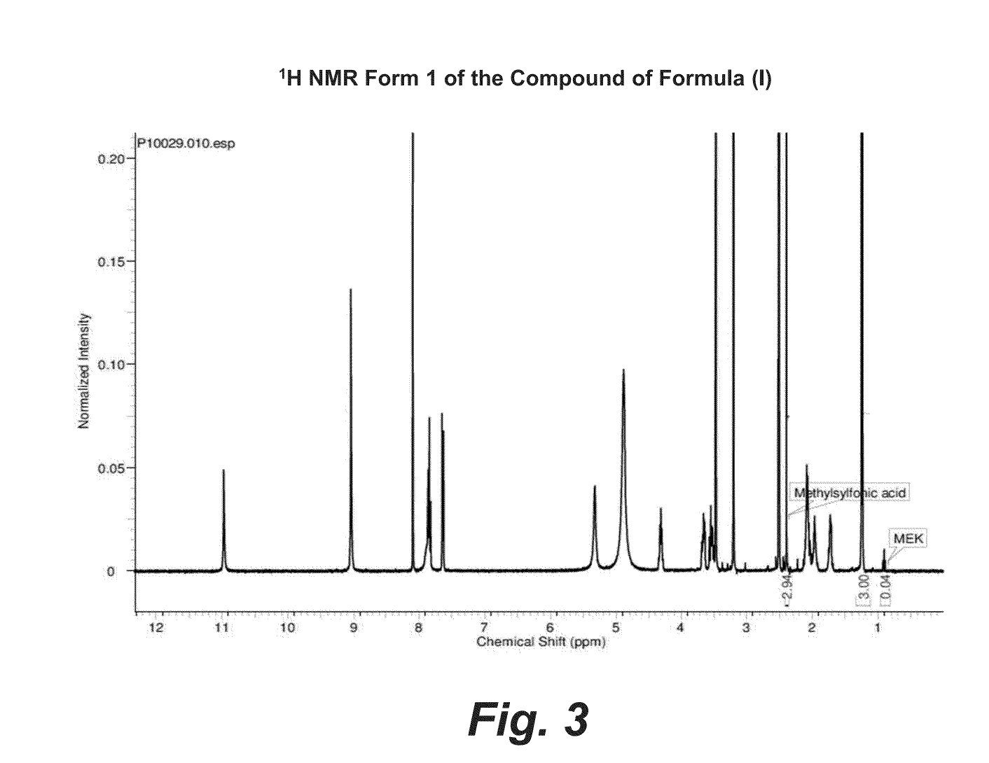

Solid Form 1 of the compound of Formula (I) can be identified by its characteristic XRPD pattern illustrated in FIG. 2 (Example 3) and its characteristic ¹H-NMR spectrum illustrated in FIG. 3 (Example 3).

Solid Form 1 of the compound of Formula (I) can preferably be made using a two step process detailed in Example 2. Briefly, this two step process involves a first DMSO hot filtration solution crystallization step to obtain a DMSO solvate of the compound of Formula (I). Compound of Formula (I) free base is suspended in DMSO. The suspension is heated to dissolve the compound of Formula (I) (≧94° C.). The solution is then cooled (≦89° C.) resulting in crystal formation. The resulting solid DMSO solvate of the compound of Formula (I) is used as the starting material for the second slurry-to-slurry crystallization step, which results in the formation of the Form 1 mesylate salt of the compound of Formula (I). In this step, the DMSO solvate is suspended in methyl ethyl ketone (MEK). The suspension is heated to dissolve the DMSO solvate (≧79° C.). Methsulfonic acid (MSA) (±MEK) is added and the solution was held at temperature (60-120 minutes) and then cooled (≦21° C.) resulting in the formation of solid Form 1 of the compound of Formula (I).

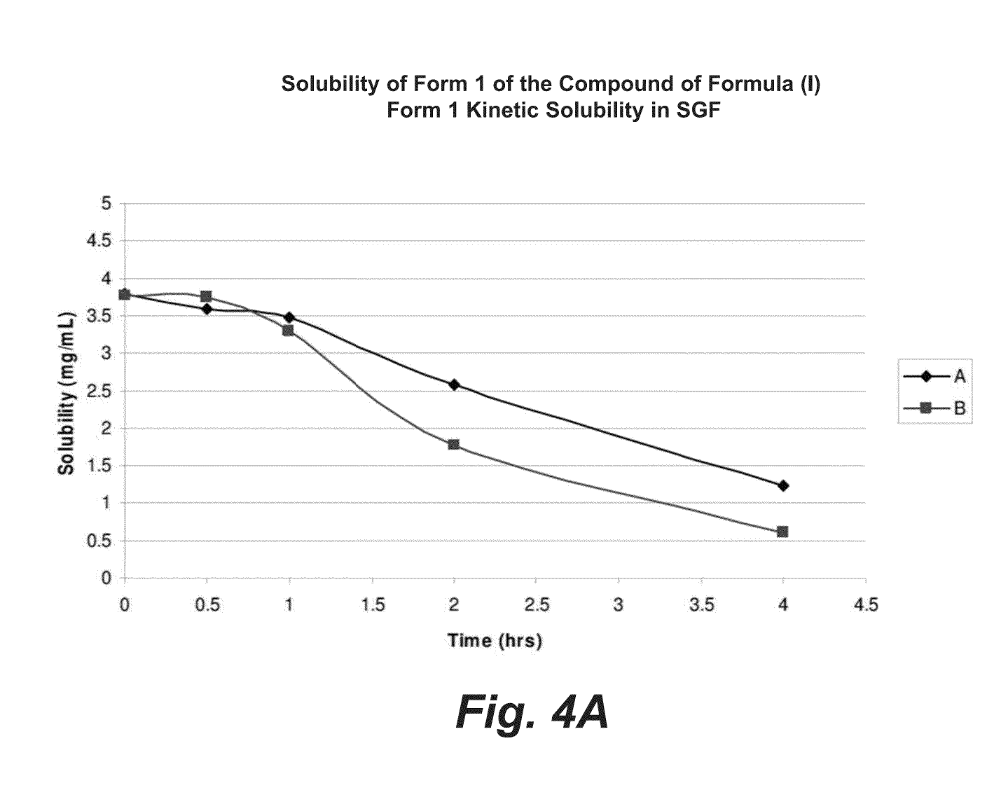

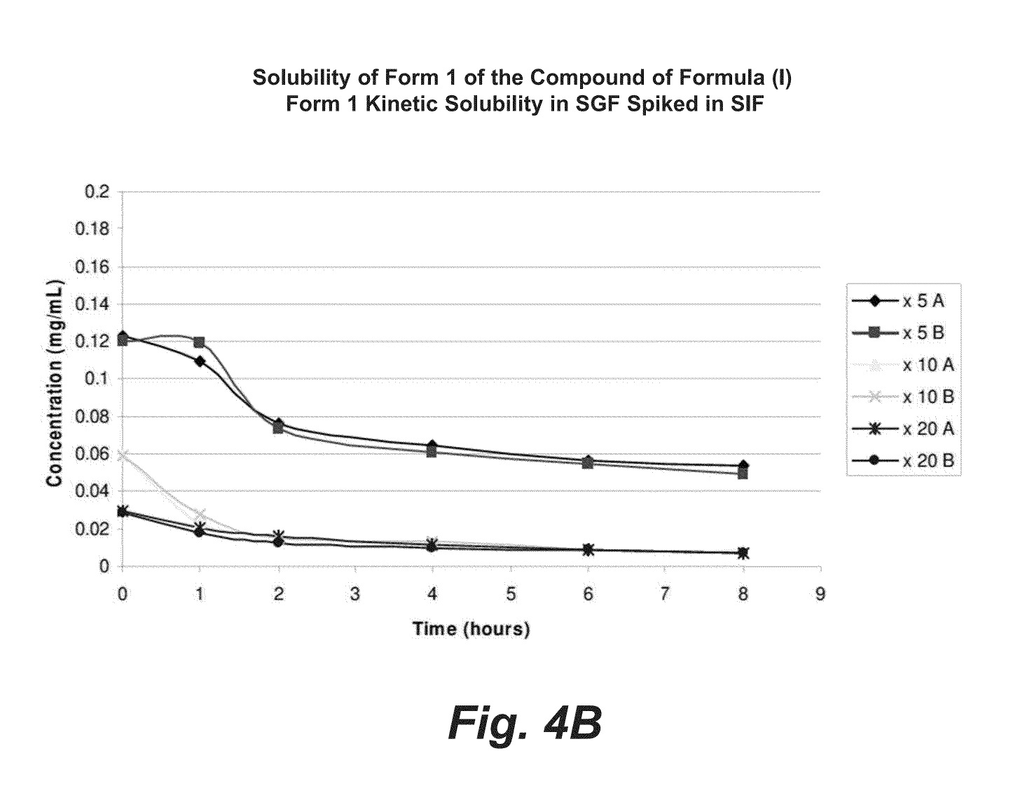

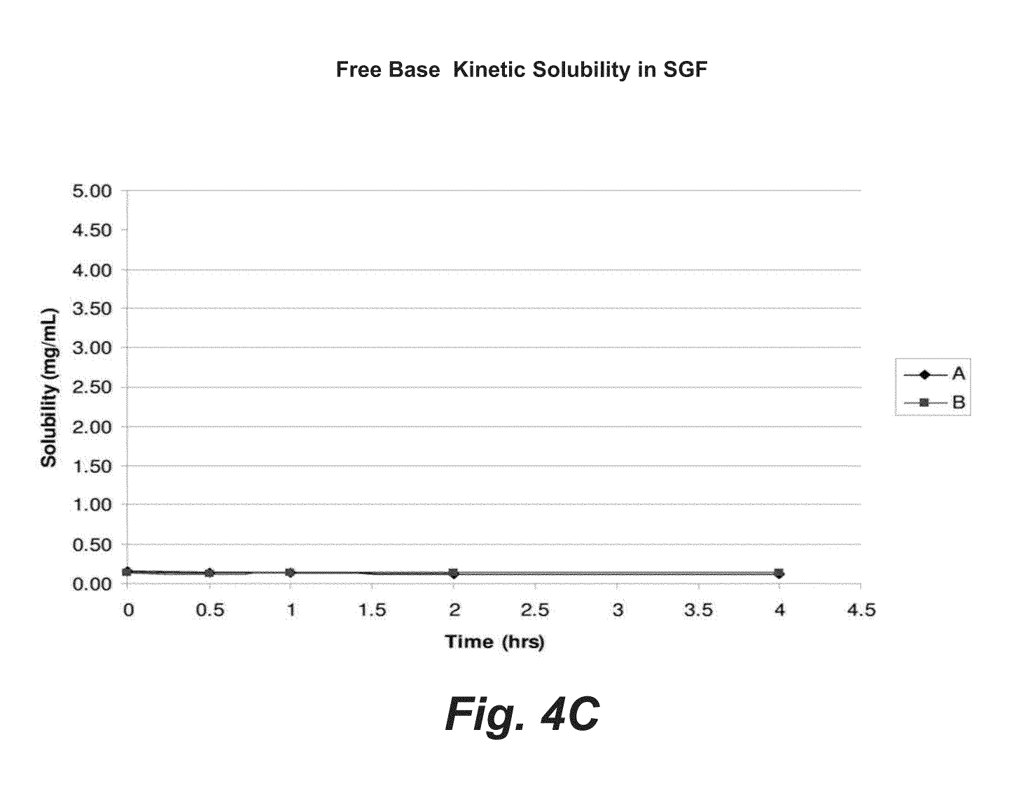

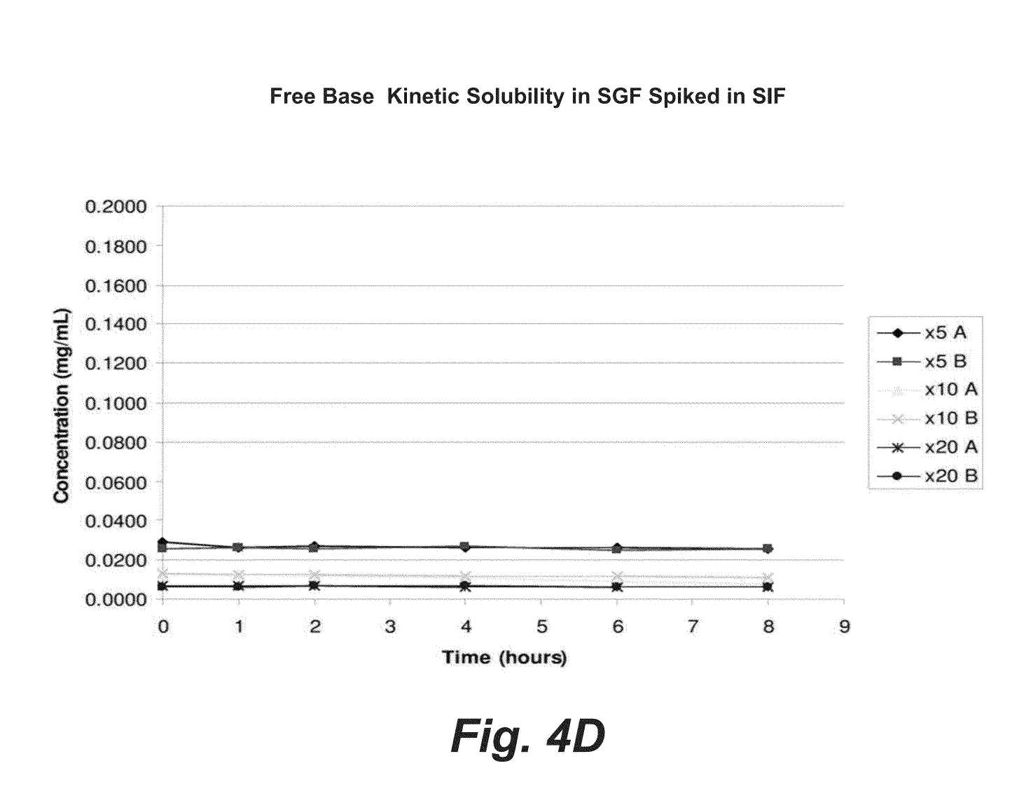

Solid Form 1 of the compound of Formula (I) also exhibits higher solubility than free base compound of Formula (I) in simulated gastric fluid (SGF) and SGF spiked simulated intestinal fluid (SIF) experiments (Example 4, FIGS. 4A-4D). Form 1 of the compound of Formula (I) of the free base compound of Formula (I) were heated in SGF to dissolve the solid in some samples the supernatant was used to spike heated SIF. HPLC was used to determine the solubility for each sample.

Solid Form 1 of the compound of Formula (I) is stable over a seven day period at room temperature, which is verified by no shift in the XRPD peaks before and after stability tests (FIGS. 4A-4D, Example 5). Stability under ambient conditions for an extended period of time is a favorable characteristic in pharmaceutical compositions.

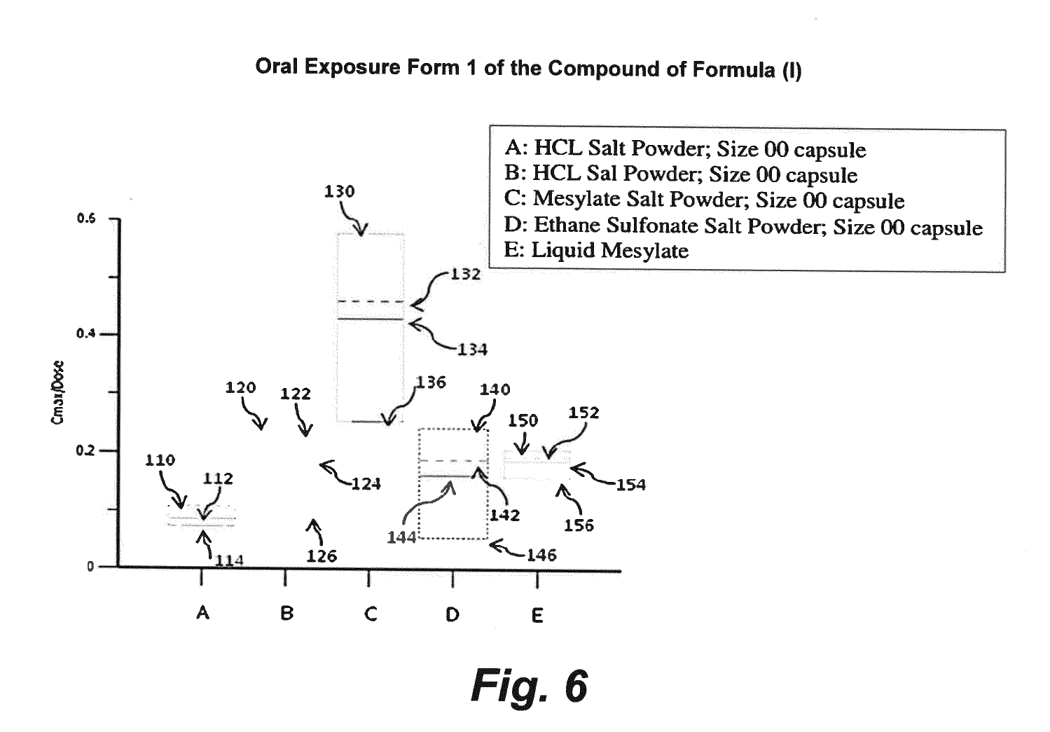

Solid Form 1 of the compound of Formula (I) also exhibits superior oral exposure (See FIG. 6). Solid Form 1 of the compound of Formula (I) was orally-administered as capsules (2 capsules at 500 mg/dose/dog) or in a size OO hard gelatin capsule (2 capsules at 400 mg/dose/dog) to dogs (n=3 dogs/dose group). Blood samples for all dose groups were collected at pre-dose, 0.25, 0.5, 1, 2, 4, 8, 12, and 24 hours and analyzed using a liquid chromatography-tandem mass spectrometry (LC/MS/MS) method. Comparison to a calibration standard demonstrated that the compound of Formula (I) Form 1 mesylate salt exhibited greater oral exposure than the comparator compounds of Formula (I) in this study.

### Formation of the Solid Form 4 of the Compound of Formula (I)

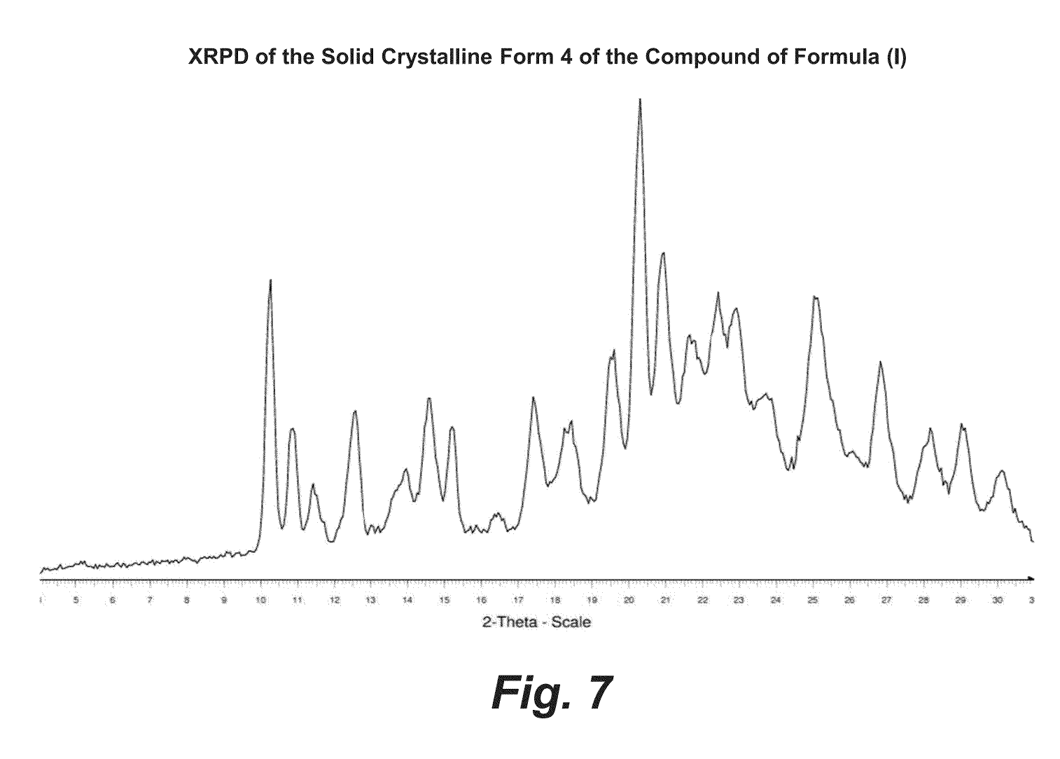

Another preferred solid crystal from of the compound of Formula (I) is the novel crystal Form 4. Solid Form 4 of the compound of Formula (I) is characterized by the XRPD pattern illustrated in FIG. 7 (Example 8). Solid Form 4 of the compound of Formula (I) can preferably be made by suspending the free base of the compound of Formula (I) in MEK and heating the suspension to dissolve the compound of Formula (I) (≧50° C.). MSA (optionally diluted in MEK) was added to the suspension of free base (79° C., ≧2 hours) and the suspension was cooled (≦25° C.) resulting in formation of the solid Form 4 of the compound of Formula (I).

### Formation of Solid Forms of the Compound of Formula (I)

Solid Form 1 and solid Form 4, as well as 13 other solid forms of the compound of Formula (I) can be made using the methods detailed below. The solid forms of the compound of Formula (I) can be identified by their characteristic XRPD patterns. These patterns are summarized (Table 6) by the 20 peaks greater than 30% intensity.

The solid forms of the compound of Formula (I) can be made using a variety of methods (Example 9). The starting material used for these methods is either the free base of the compound of Formula (I) obtained according to the procedure of Example 1 or an amorphous form of the compound of Formula (I). To obtain an amorphous starting material, the compound of Formula (I) is dissolved or suspended in a solvent (e.g., DCM/EtOH). The resulting solution or suspension (with or without filtration) can be dried to obtain amorphous compound of Formula (I) or it can be further processed. Further processing involves adding the solution or suspension to an anti-solvent (water, heptane, or n-heptane) resulting in precipitation of an amorphous solid of the compound of Formula (I).

Amorphous compound of Formula (I) is then suspended in a solvent (with shaking for 24 hours) resulting in the formation of a solid. Depending on the solvent used this process results in Form 1, Form 2, Hydrate 1, Hydrate 2 and Hydrate 3 of the compound of Formula (I) (Table 8). When amorphous compound of Formula (I) is suspended in different solvent systems at a lower temperature (with stirring at 5° C. for 24 hours) the solids Form 2, Hydrate 1, Hydrate 2, Hydrate 3, and Hydrate 4 are formed depending on the solvent selected (Table 10).

The compound of Formula (I) free base can also be used as a starting material in making solid crystalline forms of the compound of Formula (I). A variety of different solvent systems can be used to first dissolve or suspend the compound of Formula (I) free base. Different solid forms of the compound of Formula (I) are obtained depending on the solvent system used and the conditions of the crystallization.

The compound of Formula (I) free base can be suspended in a solvent and MsOH added (shaken for 24 hours) resulting in solid formation. The solids Form 1, Form 2, Hydrate 2, Solvate 2, and Solvate 3 of the compound of Formula (I) are made depending on the solvent selected (Table 9).

Solid forms of the compound of Formula (I) can also be made using DCM/IPA or DCM/EtOH as a solvent system, and n-heptane as anti-solvent. The compound of Formula (I) free base is suspended in different solvent systems (50° C.) and MSA is added. The anti-solvent n-heptane is added and the system is allowed to cool (RT) resulting in solid formation. The resulting solid is Form 1 or Form 2 of the compound of Formula (I) depending on the amount of heptane used (Table 12).

Alternatively, the compound of Formula (I) free base is suspended in DCM/IPA (RT) and MSA is added. The clear solution is heated to reflux and the solvent is distilled out. After evaporation (˜8 mL) the solution is seeded with Form 1 of the compound of Formula (I) to prompt crystal formation. After additional evaporation of solvent (˜2 mL), the system becomes a thick paste. IPA is then added resulting in solid formation (Table 13).

Alternatively, compound of Formula (I) free base is suspended in DCM/IPA (RT) and MSA is added. The clear solution is seeded with Form 1 of the compound of Formula (I). n-Heptane is added resulting in the formation of solid Form 2 of the compound of Formula (I). The suspension can be seeded again and heated (70° C., 2 hr-overnight) to eliminate the DCM. In both cases the sample contained the solid Form 2 of the compound of Formula (I) (Table 13). Instead of reseeding the solution with Form 1 of the compound of Formula (I), MEK can be added (30 min). At this point the sample contains Form 2 of the compound of Formula (I). If more MEK is added and the slurry stirred (70° C. for 3 hours) and then allowed to cool (RT) Form 1 of the compound of Formula (I) is formed (Table 13).

Solid forms of the compound of formula (I) can also be made using DMSO as the solvent. The compound of Formula (I) free base is suspended in DMSO (RT) and MSA is added. The clear solution is added into different solvents systems (with or without stirring at RT or 50° C.) resulting in the formation of Form 1 or Solvate 4 of the compound of Formula (I) depending on the solvent selected (Table 14, Table 15, Table 16, Table 17).

Solid forms of the compound of Formula (I) can also be made using DMSO/MIBK as the solvent. The compound of Formula (I) free base is suspended in DMSO/MIBK and MSA is added (90-100° C.). The anti-solvent MIBK was added (8-90 min) to the suspension. After the addition of anti-solvent is complete, the system is held at temperature (5 minutes), then cooled to room temperature (cooling rates 0.1-1.0° C./min) resulting in the formation of solid Form 1 of the compound of Formula (I) (Table 18, Table 19).

Rather than using the amorphous or the free base compound of Formula (I) as a starting material, variable temperature X-ray experiments (VT-XRPD) can be carried out using different crystalline forms as a starting material and in some cases, new crystalline forms can be observed (Table 11).

Fifteen novel crystal forms of the compound of Formula (I) can be made using either the amorphous compound of Formula (I), the free base compound of Formula (I), or by manipulation of certain crystal forms of the compound of Formula (I). Novel crystal forms of the compound of Formula (I) are useful in delivering compound of Formula (I) to treat pain through inhibition of TRPA1.

### Inhibiting TRPA1 with the Compound of Formula (I)

Preferred solid forms of the compound of Formula (I) can be administered or used in drug products to inhibit TRPA1. The compound of Formula (I) is a small molecule antagonist of the TRPA1 channel as demonstrated by in vitro testing. The compound of Formula (I) blocks inward currents through TRPA1 in rat, dog and human with an IC₅₀ of approximately 100 nanomolar (Table 1, data obtained according to Example 11). The antagonist effect of the compound of Formula (I) against hTRPA1 in a whole cell patch configuration was evaluated according to the method of Example 11.

TABLE 1

| | | | TESTED | | IC₅₀ Inward |
|---|---|---|---|---|---|
| | | | CONCS. | CURRENT | current |
| CHANNEL | SPECIES | COMPOUND | (nanomolar) | ACTIVATION | (nanomolar) |
| hTRPA1 | Human | Formula (I) | 10, 32, 100, 320, 1000 | 10 micromolar AITC | 93 ± 22 |
| rTRPA1 | Rat | Formula (I) | 32, 100, 320, 1000, 3200 | 10 micromolar AITC | 101 ± 8 |
| dTRPA1 | Dog | Formula (I) | 32, 100, 320, 1000 | 10 micromolar AITC | 102 ± 20 |

The compound of Formula (I) is highly selective for hTRPA1 as compared with TRP channels and voltage-gated ion channels. For example, when tested against eight different channels representing most of the ion channel families (Table 2, Example 11), none of the tested channels were reproducibly blocked or agonized by the compound of Formula (I) at physiologically relevant concentrations (e.g., 1, 3.2, 10, or 32 micromolar). Because the highest concentrations used (32 micromolar) had little effect, the actual IC₅₀ of the compound of Formula (I) for most of these channels cannot be determined. However, the compound of Formula (I) is at least 100-fold more selective for block of TRPA1 over all other tested channels (Table 2, Example 11).

TABLE 2

| | | | | | Fold |
|---|---|---|---|---|---|
| | TESTED | | | | Selectivity |
| | CONCS. | CURRENT | CURRENT | IC₅₀ | Compared to |
| CHANNEL | (micromolar) | ACTIVATION | EVALUATED | (micromolar) | TRPA1 |
| hTRPV1 | 1, 10 | 500 nanomolar | Inward (−80 mV) | >10 | >100 |
| | | Capsaicin | | | |
| hTRPV3 | 1, 3.2, 10, 32 | 30 micromolar | Inward (−80 mV) | >32 | >300 |
| | | 2-APB | | | |
| hTRPV4 | 3.2, 10, 32 | 2 micromolar | Inward (−80 mV) | 16 | ~170 |
| | | 4α-PDD | | | |
| hTRPV4 Agonist | 3.2, 10, 32 | None | Inward (−80 mV) | No Effect | N/A |
| hTRPV6 | 1, 3.2, 10, 32 | Voltage | Inward (−80 mV) | 34 | ~370 |
| hTRPC5 | 1, 10 | 80 micromolar | Inward (−80 mV) | >10 | >100 |
| | | LaCl₃ | | | |
| hTRPM8 | 1, 3.2, 10, 32 | 100 micromolar | Inward (−80 mV) | 19 | ~200 |
| | | Menthol | | | |
| hERG | 1, 10 | Voltage | Tail current (−40 mV) | >10 | >100 |
| hNaᵥ1.2 | 1, 3, 10 | Voltage | Peak Inward (0 mV) | >10 | >100 |

The compound of Formula (I) is a novel small molecule antagonist of the human TRPA1 channel as demonstrated by in vivo testing. For example, the compound of Formula (I) was active in rodent models of pain in vivo induced by direct activation of the TRPA1 channel with formalin injection.

The in vivo activity of the compound of Formula (I) was compared to the activity of a comparator compound of Formula (II). The compound of Formula (II) is a known TRPA1 inhibitor (see, e.g., U.S. Pat. No. 7,671,061) and was therefore used as a positive control. The compound of Formula (II) and methods of making and using this compound are disclosed as the TRPA1 inhibitor compound 200 in U.S. Pat. No. 7,671,061 (filed Dec. 22, 2006, issued Mar. 2, 2010).

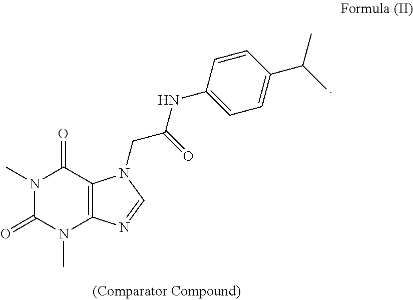 [SMILES: CC(C)c1ccc(NC(=O)Cn2cnc3c2c(=O)n(C)c(=O)n3C)cc1]

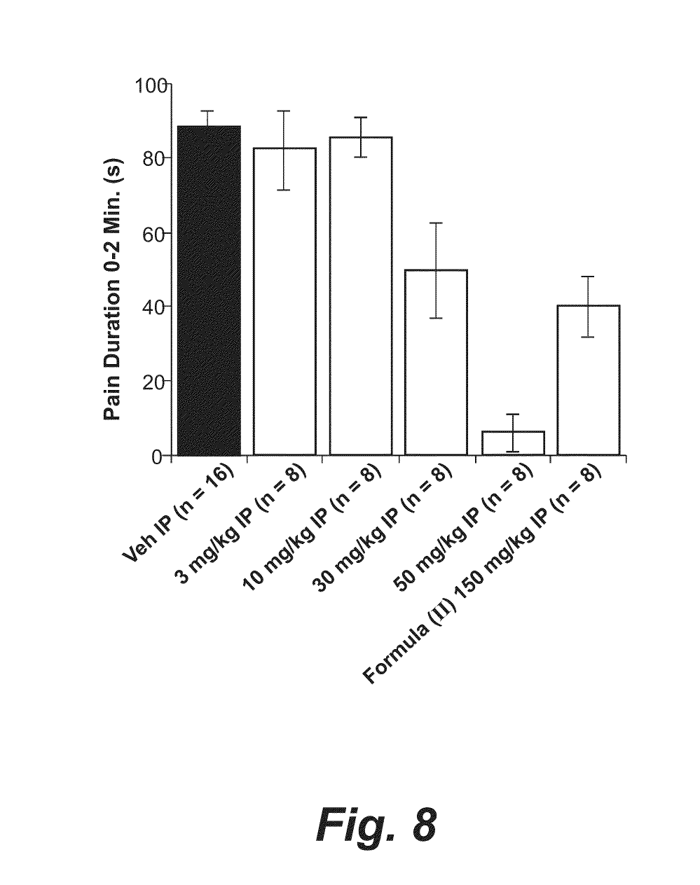

The data shown in Table 3 and FIG. 8 were obtained by administering a pharmaceutical composition comprising the compound of Formula (I) to rodents in the formalin-induced pain duration at various doses according to Example 12. Specifically, the data in Table 3 and FIG. 8 were obtained by intraperitoneal (i.p.) administration of compositions containing different concentrations of the compound of Formula (I), a comparator composition containing 150 mg/kg of the comparator compound of Formula (II) and a control composition containing the vehicle (i.e., without the compound of Formula (I) or the comparator compound of Formula (II)). As shown in Table 3 and FIG. 8, the animals treated with the compounds of Formulas (I) and (II) showed shorter durations of pain behavior than those treated with the vehicle. These data demonstrate that the compound of Formula (I) has an analgesic effect on pain caused by TRPA1 activation with formalin.

TABLE 3

| | Duration of Pain | |
|---|---|---|
| Compound and Dose | Behavior (min) | Error (min) |
| Vehicle | 88.6 | 4.3 |
| 3 mg/kg Formula (I) | 82.3 | 10.6 |
| 10 mg/kg Formula (I) | 85.8 | 5.4 |
| 30 mg/kg Formula (I) | 49.8 | 12.8 |
| 50 mg/kg Formula (I) | 5.9 | 5.0 |
| 150 mg/kg Formula (II) | 40.0 | 8.1 |

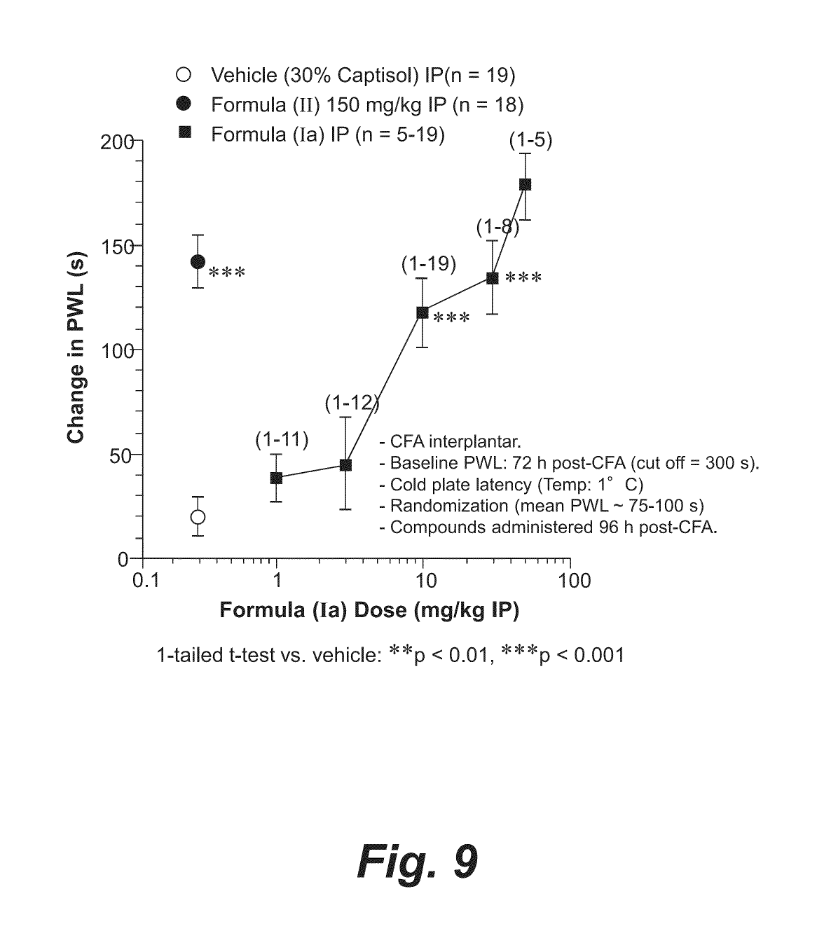

The compound of Formula (I) is also active in rodent models of pain in vivo induced by cold allodynia following chronic Complete Freund's Adjuvant-induced inflammation, as described in Example 12c. The data presented in Table 4 and FIG. 9 demonstrate increased Paw Withdrawal Latency (PWL) scores observed after i.p. administration of pharmaceutical compositions with increasing concentrations of the compound of Formula (I) in the Complete Freund's Adjuvant (CFA) rodent model described in Example 12c. This data was obtained by measuring the change in PWL score as a function of the concentration of the compound of Formula (I), as well as the PWL scores observed upon administration of a composition containing the comparator compound of Formula (II) and a control with the vehicle containing a sulfobutylether β-cyclodextrin compound (available under the tradename Captisol® from CyDex Pharmaceuticals, Inc, Lenexa, Kans.). The data shows that the compound of Formula (I) has an analgesic effect on cold allodynia.

TABLE 4

| | Change in Paw | |
|---|---|---|
| Compound and Dose | Withdrawal Latency | Error |
| Vehicle | 19.8 | 9.4 |
| 1 mg/kg Formula (I) | 38.4 | 11.5 |
| 5 mg/kg Formula (I) | 45.0 | 22.0 |
| 10 mg/kg Formula (I) | 117.6 | 16.6 |
| 30 mg/kg Formula (I) | 134.4 | 17.8 |
| 50 mg/kg/Formula (I) | 177.8 | 15.5 |
| 150 mg/kg Formula (II) | 142.2 | 12.3 |

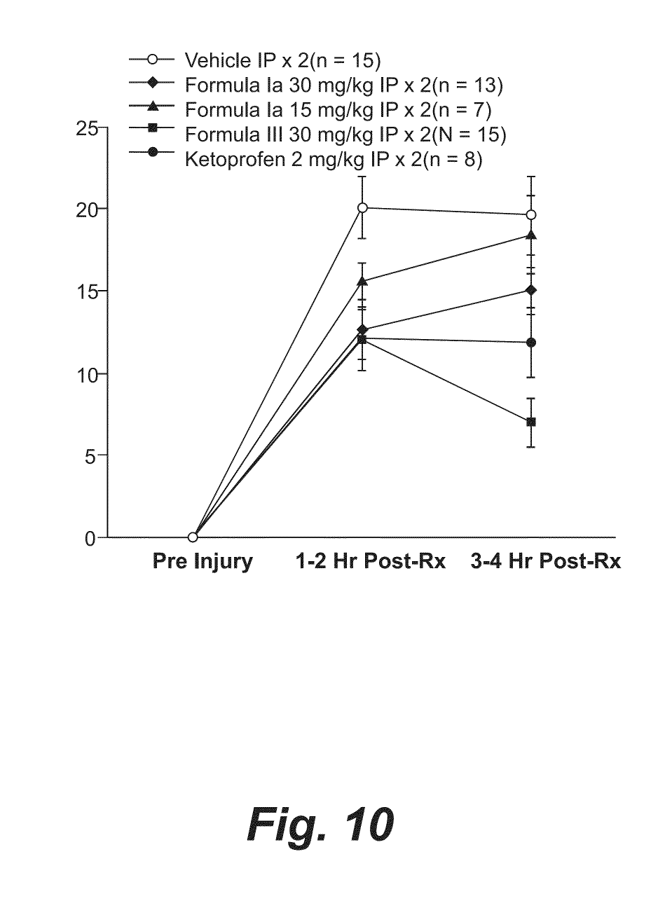

The compound of Formula (I) is also active in rodent models of pain in vivo induced by incision of the plantar surface of the hind paw (i.e., the “Brennan Surgical Model”), as described in Example 12d. FIG. 10 shows the change in guarding score as a function of the administered concentration of the compound of Formula (I), as well as the guarding scores observed upon administration of the vehicle alone and comparator pharmaceutical compositions containing the comparator compound of Formula (III), or ketoprofen. Referring to FIG. 10 and Example 12d, 60 mg/kg of the compound of Formula (I) delivered intraperitoneally (2 doses of 30 mg/kg before and immediately after the surgery) reduces spontaneous pain in the rodent incisional pain model described in Example 12d for up to 4 hours after surgery, better than ketoprofen (2 doses of 2 mg/kg intraperitoneally). Thirty (30) mg/kg of the compound of Formula (I) delivered intraperitoneally (2 doses of 15 mg/kg before and immediately after the surgery) reduces spontaneous pain for up to 2 hours after surgery (FIG. 10).

A comparator TRPA1 inhibitor of Formula (III) was also tested in the CFA rodent model of Example 12d (FIG. 10). The comparator compound of Formula (III) and methods of making and using this compound are disclosed as the TRPA1 inhibitor compound I in PCT patent application PCT/US2009/069146 (published as WO2010/075353A1 on Jul. 1, 2010).

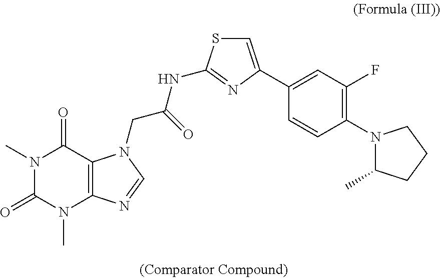 [SMILES: C[C@H]1CCCN1c1ccc(-c2csc(NC(=O)Cn3cnc4c3c(=O)n(C)c(=O)n4C)n2)cc1F]

### Pharmaceutical Compositions Comprising the Compound of Formula (I)

The compound of Formula (I) or a pharmaceutically acceptable salt thereof can be used in the manufacture of pharmaceutical compositions. Pharmaceutical compositions can be formed by combining the compound of Formula (I), or a pharmaceutically-acceptable salt thereof, with a pharmaceutically-acceptable carrier suitable for delivery to a recipient subject (e.g., a human) in accordance with a desired method of drug delivery.

Pharmaceutical compositions may be formulated for a suitable route of administration for providing the patient with an effective dosage of a compound of the present invention. For example, oral administration may be employed. Dosage forms include tablets, troches, dispersions, suspensions, solutions, capsules, patches, and the like. The most suitable formulation of a composition containing the compound of Formula (I) in any given case may depend on the severity of the condition being treated. The compositions may be conveniently presented in unit dosage form and prepared by any of the methods well known in the art of pharmacy. The compounds of Formula (I) may also be administered by controlled release means and/or delivery devices.

Pharmaceutical compositions formulated for oral delivery preferably comprise the compound of Formula (I), or a salt of the compound of Formula (I), in an amount sufficient to achieve the intended purpose (e.g., the treatment or prevention of pain or other conditions responsive to inhibition or antagonism of the TRPA1 ion channel). The amount and concentration of compound of Formula (I) in the pharmaceutical composition, as well as the quantity of the pharmaceutical composition administered to a subject, can be selected based on clinically relevant factors, such as medically relevant characteristics of the subject (e.g., age, weight, gender, other medical conditions, and the like), the solubility of the compound in the pharmaceutical composition, the potency and activity of the compound, and the manner of administration of the pharmaceutical composition. For example, a pharmaceutical composition can be formulated for oral delivery of the compound of Formula (I) (e.g., Example 10, Example 14, and Example 18).

Pharmaceutical preparations can be prepared in accordance with standard procedures selected to treat a condition that is mitigated, eliminated, prevented or otherwise treated by the administration of a compound to inhibit the TRPA1 ion channel (see, e.g., Remington's Pharmaceutical Sciences, Mack Publishing Company, Easton, Pa. and Goodman, and Gilman's “The Pharmaceutical Basis of Therapeutics,” Pergamon Press, New York, N.Y., the contents of which are incorporated herein by reference, for a general description of the methods for administering various therapeutic agents for human therapy). For example, the pharmaceutical compositions can be formulated for a desired route of administration, such as oral delivery. In particular, a medicament comprising a compound of Formula (I) can be formulated for oral administration for the therapeutic treatment of medical conditions, such as chronic or acute pain.

In preparing the compositions for oral dosage form, any of the usual pharmaceutical media may be employed as carriers, such as, for example, water, glycols, oils, alcohols, flavoring agents, preservatives, coloring agents, and the like in the case of oral liquid preparations (such as suspensions, solutions and elixirs) or aerosols; or carriers such as starches, sugars, micro-crystalline cellulose, diluents, granulating agents, lubricants, binders, disintegrating agents, and the like may be used in the case of oral solid preparations such as, for example, powders, capsules, and tablets, with the solid oral preparations being preferred over the liquid preparations. An example of a solid oral preparation is tablets or capsules containing the compound of Formula (I). If desired, tablets may be coated by standard aqueous or non-aqueous techniques.

In one embodiment, provided herein is a pharmaceutical composition comprising a lactose, one or more cellulose polymers, and the mesylate salts provided herein. In an embodiment, the pharmaceutical composition is suitable for oral administration. In one embodiment of the composition, the mesylate salt is an amorphous mesylate salt of the compound of Formula (I). In another embodiment of the composition, the mesylate salt is a crystalline mesylate salt of the compound of Formula (I). In yet another embodiment of the composition, the mesylate salt is a crystalline mesylate salt of the compound of Formula (I) characterized by an X-ray powder diffraction pattern with peaks defined in units of 2-theta±0.3 (2θ±0.3°) at 11.0, 12.5, 13.7, 14.8, 17.6, 22.3, 23.2 26.0, and 28.7. In still another embodiment of the composition, the mesylate salt is a crystalline mesylate salt of the compound of Formula (I) characterized by an X-ray powder diffraction pattern with peaks (2θ±0.3°) at 4.8, 15.8, 17.8, 18.7, 21.2, 23.6, and 24.1. In another embodiment of the composition, the mesylate salt is a crystalline mesylate salt of the compound of Formula (I) characterized by an X-ray powder diffraction pattern with peaks (2θ±0.3°) in Table 6.

The cellulose polymers are pharmaceutically acceptable cellulose polymers. See, e.g., the U.S. Food and Drug Administration's Database of Select Committee on GRAS Substances (SCOGS) Reviews. In one embodiment of the pharmaceutical composition suitable for oral administration, the cellulose polymers are one or more of microcrystalline cellulose, croscarmellose sodium, hydroxymethyl cellulose, hydroxyethyl cellulose, hydroxypropyl cellulose, hydroxypropyl methyl cellulose acetate succinate and methyl cellulose. In another embodiment, the cellulose polymers are microcrystalline cellulose and croscarmellose sodium. In a particular embodiment, the pharmaceutical composition comprises both microcrystalline cellulose and croscarmellose sodium.

In another embodiment of the pharmaceutical composition suitable for oral administration, the lactose is selected from the group consisting of lactose monohydrate and lactose anhydrous. In a particular embodiment, the lactose is lactose monohydrate.

The components of the oral administration can make up various percentages of the total composition. For example, in one embodiment, the lactose is 15-35%, the cellular polymers are 13-33%, and the mesylate salt is 40-60% by weight of the pharmaceutical composition. In another embodiment, the composition comprises 25 weight % lactose monohydrate, 7 weight % microcrystalline cellulose, 16 weight % croscarmellose sodium, and 50 weight % mesylate salt.

The composition can further include additional pharmaceutically acceptable components. The additional components can comprise, for example, 0.1-5% by weight of the pharmaceutical composition. For example, in one embodiment, the pharmaceutical composition further comprises colloidal silicon dioxide or magnesium stearate.

The pharmaceutical composition for oral administration provided above can be in the form of a spray-dried dispersion.

The pharmaceutical composition for oral administration provided above can be in the form of a tablet.

A non-limiting example of a pharmaceutical composition suitable for compression into a tablet for oral administration is provided below as Table 5.

TABLE 5

| Ingredients | Function | % weight |
|---|---|---|
| Crystal Mesylate Salt | Active ingredient | 50 |
| Lactose Monohydrate | Ductile Filler | 25 |
| (310 NF grade) | | |
| Microcrystalline Cellulose | Binder | 7 |
| (Avicel PH101) | | |
| Croscarmellose Sodium | Disintegrant | 8 |
| (Ac-di-sol) | | |
| Colloidal Silicon Dioxide | Glidant | 0.5 |
| (Cab-o-sil) | | |
| Magnesium Sterate | Lubricant | 0.5 |

Extragranular Ingredients

| | | |
|---|---|---|
| Croscarmellose Sodium | Disintegrant | 8 |
| (Ac-di-sol) | | |
| Colloidal Silicon Dioxide | Glidant | 0.5 |
| (Cab-o-sil) | | |
| Magnesium Sterate | Lubricant | 0.5 |

The pharmaceutical compositions comprising one or more compounds of Formula (I) can be sterilized, for example, by filtration through a bacteria-retaining filter, or by incorporating sterilizing agents in the form of sterile solid compositions, which can be dissolved or dispersed in sterile water or other sterile injectable medium just prior to use.

### Administration of Compositions Comprising the Compound of Formula (I)

Pharmaceutical compositions containing the compound of Formula (I) or pharmaceutically acceptable salts thereof can be used to treat or ameliorate medical conditions responsive to the inhibition of the TRPA1 ion channel in subjects (e.g., humans and animals). For example, the pharmaceutical compositions comprising a compound of Formula (I), or a pharmaceutically acceptable salt thereof, are useful as a perioperative analgesic, for example in the management of mild to moderate acute post-operative pain and management of moderate to severe acute pain as an adjunct to opioid analgesics. The pharmaceutical compositions comprising a therapeutically-effective dose of the compound of Formula (I) can be administered to a patient for treatment of pain in a clinically safe and effective manner, including one or more separate administrations of the pharmaceutical compositions comprising the compound of Formula (I). For example, a pharmaceutical composition, when administered to a subject, results in an ALT and/or AST level of less than about 250 mg/dL (e.g., about 200 mg/dL, 150 mg/dL, 100 mg/dL or 50 mg/dL) three days after administration.

The amount of active ingredients which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about fifty percent of active ingredient. In one embodiment, this amount is 1.6% (weight to weight). In another embodiment, this amount is 40% (weight to volume). Pharmaceutical compositions can contain, for example, 1 to 50% of a compound of Formula (I) in combination with a pharmaceutically acceptable carrier.

Pharmaceutical compositions containing the compound of Formula (I) or pharmaceutically acceptable salts thereof can be used to treat or ameliorate pain. Methods of treating medical conditions responsive to the inhibition of the TRPA1 ion channel in subjects (e.g., humans and animals) can include the administration of a therapeutically effective amount of the compound of the Formula (I) or a pharmaceutically-acceptable salt thereof. The pain can be chronic or acute. Methods of treatment can include administering to a subject in need thereof a therapeutically-effective amount of the compound of Formula (I) or a pharmaceutically acceptable salt thereof in one or more doses over a course of treatment. The pharmaceutical compositions comprising a therapeutically-effective dose of the compound of Formula (I) can be administered to a patient for treatment of pain in a clinically safe and effective manner, including one or more separate administrations of the pharmaceutical compositions comprising one or more compounds of Formula (I).

Pharmaceutical compositions comprising a compound of Formula (I) (e.g., a compound of Formula (I) are useful for administration for the treatment of respiratory conditions. Such conditions affect the lung, pleural cavity, bronchial tubes, trachea, upper respiratory tract as well as the nerves and muscles involved in breathing.

A method for treating or ameliorating asthma in an animal or human, comprising administering to the animal or human a pharmaceutical composition comprising a therapeutically effective amount of a compound of Formula (I), or a pharmaceutically acceptable salt thereof, by inhalation. In one example of this method, the compound of Formula (I) is in the form of a mesylate salt. The asthma can be allergic asthma. The pharmaceutical composition can be administered as an aerosol. The pharmaceutical composition is administered using a medical nebulizer.

The mesylate salt of the compound of Formula (I) is useful for the treatment or amelioration of respiratory and/or pulmonary indications that are therapeutically responsive to administration of a compound of Formula (I), or other TRPA1 inhibitor compounds. Pharmaceutical compositions comprising a compound of Formula (I) can be administered to the lung for treatment of respiratory and/or pulmonary indications. According to the invention, the inhalable compositions comprising a compound of Formula (I) can be delivered by any suitable inhalation device that is adapted to administer a controlled amount of such a pharmaceutical composition to a patient. Suitable inhalation devices may rely upon the aerosolisation energy of the patient's own breath to expel and disperse the dry powder dose. Alternatively, this energy may be provided by an energy source independent of the patient's inhalation effort, such as by impellers, patient/device created pressurized gas sources or physically (e.g. compressed gas) or chemically stored energy sources.

Pharmaceutical compositions comprising a formulation containing the compound of Formula (I) can be characterized by particles of a therapeutically effective size and shape. Particles containing the compound of Formula (I) for administration by inhalation desirably have an aerodynamic diameter permitting a patient to inhale and retain the particles at the therapeutically relevant site within the lung. For some indications, a particle size of greater than about 1 micron is desirable, to permit retention of the particle within the respiratory tract. A particle size of about 2-4 microns is suitable for treatment of certain indications (e.g., COPD), while a particle size of about 8 microns can be suitable for other indications (e.g., cough). In general, the particles containing the compound of Formula (I) for administration by inhalation desirably have an aerodynamic diameter of from 1-10 microns, preferably from about 2 to about 8 microns. If necessary, the size of particles obtained by crystallization may conveniently be reduced by micronization.

The term “aerodynamic particle size” is defined for the purposes of the present application as the diameter of a sphere of unit density which has the same settling velocity in air as the aerosol particle being measured (e.g., measured as an analytical parameter using a Cascade Impactor (CI)). Aerodynamic diameter is measured by a cascade impactor. The term “mass median aerodynamic diameter” or “MMAD” is defined as the median of the distribution of mass with respect to aerodynamic diameter. The median aerodynamic diameter and the geometric standard deviation are used to describe the particle size distribution of an aerosol, based on the mass and size of the particles. According to such a description, fifty percent of the particles by mass will be smaller than the median aerodynamic diameter, and fifty percent of the particles will be larger than the median aerodynamic diameter.

Pharmaceutical compositions comprising a formulation containing the compound of Formula (I) formulated for administration by inhalation can be delivered with a dry powder inhaler device. Dry powder inhalers (DPI's) are well known devices for administering pharmaceutically active agents to the respiratory tract. Consequently, they are particularly suitable when used for the administration of active agents in the treatment of diseases such as asthma, bronchitis, cough, chronic obstructive pulmonary disease (COPD), emphysema, rhinitis, etc. Since the drug acts directly on the target organ much smaller quantities of the active ingredient may be used, thereby minimizing any potential side effects. Dry powder compositions for use as inhalable medicaments in DPI's typically comprise a pharmaceutically active agent intimately admixed with an excess of pharmaceutically acceptable excipient or excipients (often called carrier(s)). Such excipients serve not only to dilute the quantity of active agent administered in each dose but also to establish acceptable manufacture of the powder mixture and aid in the aerosolisation of the drug. Such a high proportion of excipient will essentially determine the properties of the powder formulation, particularly the manufacturing characteristics.

Accordingly, in one embodiment, provided herein is a pharmaceutical composition in the form of a dry powder for inhalation, comprising the mesylate salts provided herein. In one embodiment of the composition, the mesylate salt is an amorphous mesylate salt of the compound of Formula (I). In another embodiment of the composition, the mesylate salt is a crystalline mesylate salt of the compound of Formula (I). In yet another embodiment of the composition, the mesylate salt is a crystalline mesylate salt of the compound of Formula (I) characterized by an X-ray powder diffraction pattern with peaks defined in units of 2-theta±0.3 (2θ±0.3°) at 11.0, 12.5, 13.7, 14.8, 17.6, 22.3, 23.2 26.0, and 28.7. In still another embodiment of the composition, the mesylate salt is a crystalline mesylate salt of the compound of Formula (I) characterized by an X-ray powder diffraction pattern with peaks (2θ±0.3°) at 4.8, 15.8, 17.8, 18.7, 21.2, 23.6, 24.1. In another embodiment of the composition, the mesylate salt is a crystalline mesylate salt of the compound of Formula (I) characterized by an X-ray powder diffraction pattern with peaks (2θ±0.3°) in Table 6.

The dry powder form can comprise the mesylate salt in particle form. For example, the particles can have a particle size distribution such that 50% of the particles are smaller than 8 μm, e.g., a particle size distribution such that 50% of the particles are smaller than 6.8 μm. The particles can also have a particle size distribution such that 50% of the particles are smaller than 4 μm, e.g., a particle size distribution such that 50% of the particles are smaller than 2.7 μm.

The particles can be produced by any number of known techniques (e.g., micronization). In an embodiment, provided herein is a process for preparing particles of the mesylate salts described herein (e.g., in crystalline or amorphous form), the method comprising the steps of dissolving the mesylate salt in n-heptane or ethyl acetate, followed by spray drying to form the particles. In an embodiment, the formed particles have a particle size distribution such that 50% of the particles are smaller than 1-10 μm. In another embodiment of the production method, the particles have a particle size distribution such that 50% of the particles are smaller than 8 μm or smaller than 4 μm.

The dry powder form comprising a compound of Formula (I) can be administered to the lung for treatment of respiratory and/or pulmonary indications. In an embodiment, provided herein is a method of treating asthma, a cough or COPD in a subject in need thereof, comprising administering to a subject in need thereof an effective amount of the dry powder forms described above.

In one example, the compound of Formula (I) can be orally administered to a subject human. The total daily dose of a compound of Formula (I) can be about 0.1 mg/kg/day to about 50 mg/kg/day of the compound of Formula (I) administered orally to a subject one to four times a day (e.g., QD, BID, TID, or QID). The total daily dose administered to a human can also be about 1 mg/kg/day to about 25 mg/kg/day, or about 3 mg/kg/day to about 10 mg/kg/day. The amount per administered dose or the total amount administered will depend on such factors as the nature and severity of the pain, the age and general health of the patient, and the tolerance of the patient to the compound. For example, a pharmaceutical composition formulated for oral delivery can contain one or more solid forms of a compound of Formula (I) for administration to a patient in need thereof in a daily dose of about 500-1500 mg one to four times per day (including, e.g., doses of about 500-600 mg once, twice, three or four times per day).

For example, a pharmaceutical composition comprising a therapeutically effective dose of the compound of Formula (I), or a pharmaceutically acceptable salt thereof, can be administered (e.g., orally) to a subject in need thereof multiple times per day (e.g., BID) over a course of treatment of one or more days to treat pain in the subject.

### EXAMPLES

Certain examples below illustrate the synthesis of the compound of Formula (I) and pharmaceutically acceptable salts thereof. Further, the disclosure includes variations of the methods described herein to produce the compounds of Formula (I) that would be understood by one skilled in the art based on the instant disclosure.

### Example 1A

### Synthesis of the Compound of Formula (I) Free Base

### Step 1

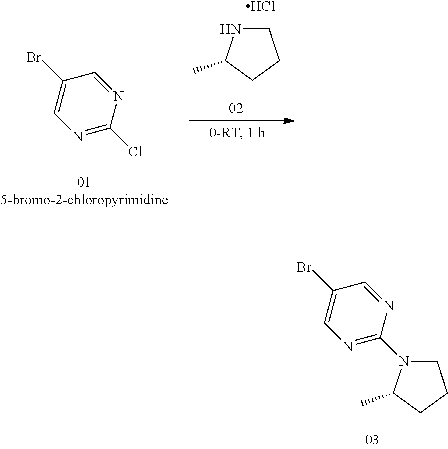

A dry 1 L round bottom flask charged with (S)-2-methylpyrrolidine (compound 02) (44.2 mL, 465 mmol) was cooled to 0° C. Compound 01 (60 g, 310 mmol) was added to the cooled amine compound 02 over 2 minutes (observed extreme exotherm). After addition was complete, the reactants were warmed to room temperature and continued to stir for 1 hr. The reaction was followed by LCMS and UPLC.

The resulting orange solids were dissolved in (200 mL 9:1 DCM:MeOH), washed with sat. sodium bicarbonate 150 mL, water (3×100 mL). The combined aqueous layers were back extracted with (9:1 DCM:MeOH). The combined organic layers were washed with brine, dried over MgSO₄, and concentrated onto silica. The reaction was column purified using 400 g silica column w/(Hex:EtOAc) solvent system (0% 4CV; 0-30% 6 CV; 30% 6 CV). The product was eluted between 20-30% EtOAc. The fractions containing product were combined and dried under vacuum, and the resulting clear oil was treated with hexanes, agitated, then evaporated. Fine crystal formation was. The residue was allowed to stand at 0° C. to aide white crystalline solids of compound 03.

For compound 03 in Step 1, Example 1: Isolated Yield: 67.2 g (89%) as white crystalline solids. (m/z M+=241); ¹H NMR (300 MHz, DMSO) δ 9.01 (s, 1H), 8.42 (s, 2H), 4.20-4.06 (m, 1H), 3.56-3.34 (m, 2H), 2.12-1.81 (m, 3H), 1.68 (s, 1H), 1.16 (d, J=6.3 Hz, 3H).

### Step 2

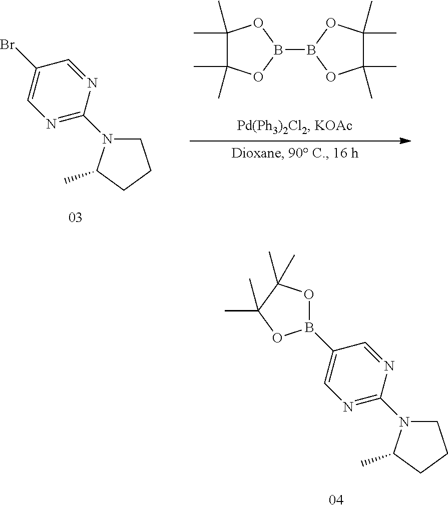

A 2 L three neck round bottom flask was charged with compound 03 (45 g, 186 mmol), Bis(pinacolato)diboron (65.2 g, 257 mmol), bis(triphenylphosphine)palladium chloride (13.05 g, 18.59 mmol), Potassium acetate (36.5 g, 372 mmol) and suspended in anhydrous Dioxane (Volume: 929 mL). The flask was flushed with nitrogen, fitted with a reflux condenser and heated to 90° C. overnight.

The Dioxane was removed in vacuo. The crude material was dissolved in (200 mL) DCM and washed with water (3×100 mL). The combined aqueous layers were back extracted with EtOAc. The combined organic layers were washed with brine, dried over MgSO4, and concentrated onto silica. The material was split into two batches and column purified using 200 g silica column with Hexane:EtOAc solvent system (0% CV; 3% 8 CV; 5-20% 10 CV; 20-50% 5 CV). The starting material eluted with 3% EtOAc while the desired product was eluted between 5-40% EtOAc. Fractions containing product were combined and solvent was removed in vacuo to afford compound 04.

For compound 04 in Step 2, Example 1: Isolated Yield: 23.0 g (42%) as off-white solids. [(m/z=M+=289.20 (boronic acid observed at m/z 207.12)); ¹H NMR (300 MHz, DMSO) δ 8.45 (s, 2H), 4.31-4.17 (m, 1H), 3.62-3.38 (m, 2H), 2.12-1.81 (m, 3H), 1.73-1.61 (m, 1H), 1.27 (s, 12H), 1.17 (d, J=6.3 Hz, 3H).

### Step 3

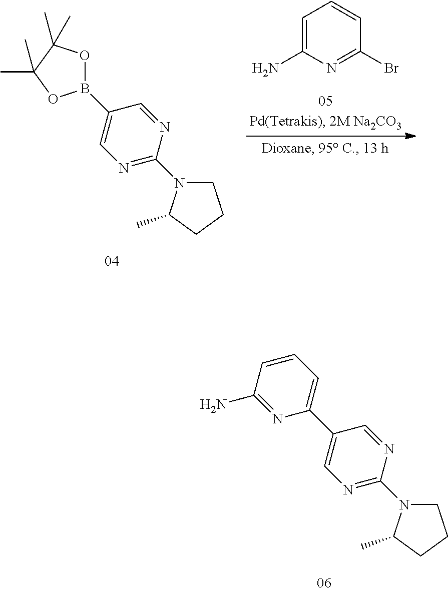 [SMILES: C[C@H]1CCCN1c1ncc(B2OC(C)(C)C(C)(C)O2)cn1 ; Nc1cccc(Br)n1 ; C[C@H]1CCCN1c1ncc(-c2cccc(N)n2)cn1]

A 1 L round bottom flask was charged with compound 05 (15.14 g, 87 mmol) and compound 04 (23.00 g, 80 mmol), purged with nitrogen, followed by addition of Pd(Ph₃P)₄ (9.19 g, 7.95 mmol). The solids were suspended in a mixture of anhydrous Dioxane (398 mL) and aqueous 2M sodium carbonate (119 mL, 239 mmol). The reaction was heated to 95° C. for 13 hrs.

The organics were separated from salts by transfer of liquid phase to 2 L round bottom. The salts were rinsed with dioxane and combined with the previously separated dioxane solution. The dioxane was removed under vacuo. The yellow crude residue was dissolved in DCM and washed with water (3×100 mL), brine, dried over MgSO₄ and concentrated onto silica, purified by column chromatography using 200 g silica column w/DCM:EtOAc solvent system (0% 20 CV; 20% 10 CV; 50-80% 10 CV; 80% 5 CV). The desired product eluted between 50-80% EtOAc. Fractions containing product were concentrated to isolate the compound 06.

For compound 06 in Step 3, Example 1: Isolated Yield: 13.7 g (67%) as light yellow solids. (m/z=M+=255.15); ¹H NMR (300 MHz, DMSO) δ 8.88 (s, 2H), 7.40 (t, J=7.8 Hz, 1H), 6.95 (d, J=7.1 Hz, 1H), 6.35 (d, J=7.9 Hz, 1H), 5.96 (s, 2H), 4.31-4.19 (m, 1H), 3.66-3.41 (m, 2H), 2.13-1.84 (m, 3H), 1.75-1.65 (m, 1H), 1.22 (d, J=6.3 Hz, 3H).

### Step 4

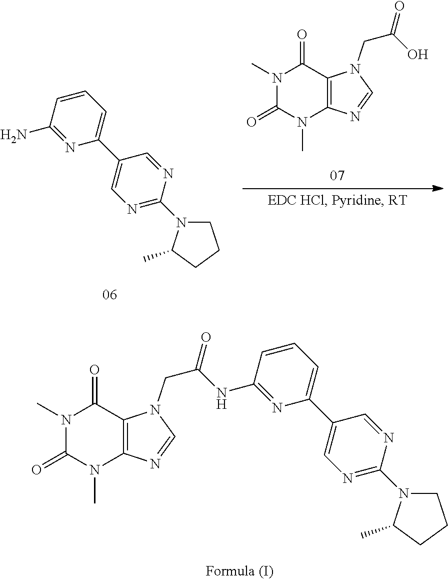 [SMILES: C[C@H]1CCCN1c1ncc(-c2cccc(N)n2)cn1 ; Cn1c(=O)c2c(ncn2CC(=O)O)n(C)c1=O ; C[C@H]1CCCN1c1ncc(-c2cccc(NC(=O)Cn3cnc4c3c(=O)n(C)c(=O)n4C)n2)cn1]

A dry 200 mL round bottom was charged with compound 07 (12.17 g, 51.1 mmol), compound 06 (13.7 g, 53.7 mmol), EDC (19.59 g, 102 mmol) flushed with nitrogen followed by addition of anhydrous Pyridine (Volume: 128 mL) (no exotherm observed). The suspension was stirred at room temperature for 1 h.

The reaction mixture was diluted with 100 mL water and an off-white precipitate was observed. Transferred suspension to 500 mL flask charged with stir bar and diluted with 150 mL 0.1M HCl while stirring. Observed precipitate turn light red in color forming an amorphous solid. Extracted aqueous with EtOAc (3×100 mL). The organic layer was washed with 0.1M HCl (3×50 mL), water, brine, dried over MgSO₄ and concentrated onto silica. Column purified with using DCM:MeOH solvent system (0% 5 CV; 0-3% 10 CV; 3-4% 4 CV; 4% 10 CV). Product eluted between 3-4% MeOH. Pooled appropriate fractions removed solvents in vacuo, placed on high vacuum to afford the compound of Formula (I).

For the compound of Formula (I) in Step 4, Example 1: Isolated Yield: 20.7 g (85%) as off-white solids. The compound Formula (I) (m/z=M+=475), ¹H NMR (300 MHz, DMSO) δ 10.95 (s, 1H), 9.01 (s, 2H), 8.09 (s, 1H), 7.82 (t, J=7.6 Hz, 2H), 7.61 (d, J=8.4 Hz, 1H), 5.32 (s, 2H), 4.33-4.23 (m, 1H), 3.71-3.49 (m, 2H), 3.47 (s, 3H), 3.20 (s, 2H), 2.18-1.84 (m, 3H), 1.70 (m, 1H), 1.24 (d, J=6.3 Hz, 3H).

### Example 1B

### Synthesis of Deuterated Compound of Formula (I)

A deuterated compound (12) was prepared as described in FIG. 1B. Compound 10 was prepared from a commercial starting material compound 08 according to the following procedure:

Suspended Theophiline-d6 (0.480 g, 2.58 mmol), potassium carbonate (0.392 g, 2.84 mmol), in DMF (Volume: 12.89 mL), followed by addition of ethyl 2-chloroacetate (0.275 mL, 2.58 mmol) and heated to 90° C. for 1 hr. Cooled reaction mixture to room temperature and diluted into 15 mL stirred water solution at room temperature. To the aqueous solution added lithium hydroxide (0.123 g, 5.16 mmol) in 10 mL water continued to stir at room temperature for 1 hr. Titrated solution to pH 4 with 5M HCl aq. The resulting white solids were collected via vacuum filtration to afford compound 10 (0.510 g, 81%) ESI-MS (EI+, m/z): 244.11

Deuterated compound 12 was synthesized in the same manner as formula (I) using compound 06 (0.150 g, 0.609 mmol), and compound 10 (0.163 g, 0.640 mmol). The resulting crude solids were collected via vacuum filtration. Column purified by silica gel chromatography to afford deuterated compound 12 (0.135 g, 46%) ESI-MS (EI+, m/z): 481.25. ¹H NMR (300 MHz, DMSO) δ 10.95 (s, 1H), 9.01 (s, 2H), 8.08 (s, 1H), 7.82 (t, J=7.7 Hz, 2H), 7.61 (d, J=8.5 Hz, 1H), 5.76 (s, 1H), 5.32 (s, 2H), 4.29 (s, 1H), 3.69-3.56 (m, 1H), 3.53 (s, 1H), 2.13-1.85 (m, 3H), 1.71 (d, J=2.3 Hz, 1H), 1.24 (d, J=6.3 Hz, 3H).

In addition to compound 12, the compounds described herein also include isotopes of the compound of Formula (I). For example, isotopes of Formula (I) can be formed as molecules formed by substitution of atomic isotopes at one or more of the atoms that constitute the compound of Formula (I). For example, the isotopes of Formula (I) may be radiolabeled with radioactive isotopes. Isotopes of Formula (I) include compounds formed by substitution of hydrogen in Formula (I) with deuterium (²H), or tritium (³H), or substitution of one or more carbon atoms in Formula (I) with carbon-13 (¹³C) or carbon-14 (¹⁴C). Preferred isotopes of Formula (I) inhibit TRPA1 in humans or animals. All isotopic variations of the compounds disclosed herein, whether radioactive or not, are intended to be encompassed within the scope of the present invention. For example, deuterated compounds or compounds containing ¹³C are intended to be encompassed within the scope of the invention.

### Example 2

### Formation of Solid Form 1 of a Compound of Formula (I)

The solid Form 1 of the compound of Formula (I) was made through a two step process. The first step is a DMSO hot filtration solution crystallization step and the second is a slurry-to-slurry crystallization step.

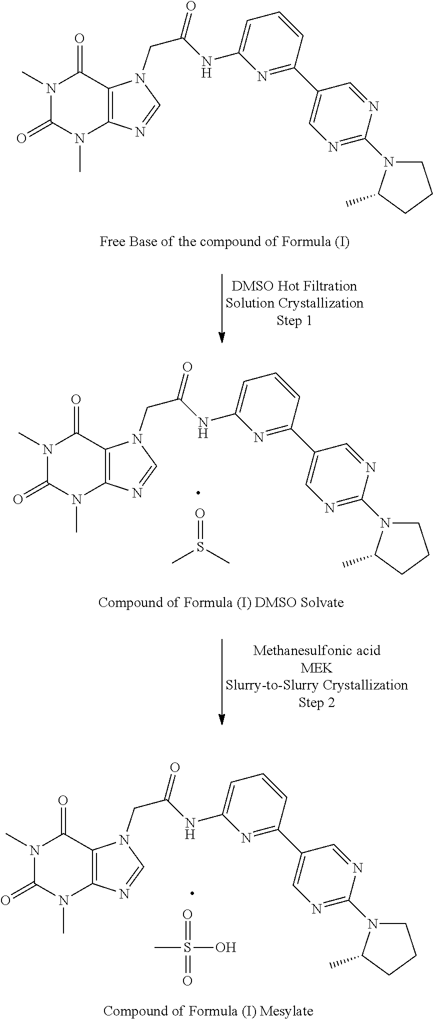

### Preparation of Free Base DMSO Solvate (Step 1)

The compound of Formula (I) free base (202 g) and 1600 mL of DMSO was charged to a reactor sequentially. The suspension was stirred at 100 rpm and heated to 94° C. under N2 stream at 4° C./min. The suspension turned to clear solution at 94° C. The hot solution was filtered through 0.7 μm filter, and cooled to 21° C. at 0.2° C./min with stirring at 100 rpm. The crystallization started at ˜83° C. After filtration, the cake was washed with MEK (350 mL twice) and vacuum dried at RT for 2 hour to afford white needles (209 g).

Alternative 1:

Compound of Formula (I) free base (5 g) and 40 mL of DMSO was charged to the reactor sequentially. The suspension was stirred at 300 rpm and heated to 96° C. under N2 stream at 2° C./min. The suspension turned to a clear solution at 94° C. The hot solution was filtered through 0.7 μm membrane filter, and then cooled to 16° C. at 300 rpm and a cooling rate of 2° C./min. The crystallization started at 80° C. After filtration, the cake was washed with MEK (20 mL twice) and vacuum dried at RT for 1 hour to afford light pink crystalline (6.94 g).

Alternative 2:

Compound of Formula (I) free base (5.72 g) and 25 mL of DMSO was charged to the reactor sequentially. The suspension was stirred at 300 rpm and heated to 160° C. at a heating rate of 10° C./min. The suspension turned to a clear solution at 120° C. After 20 min, the solution turned to brown at 150° C. The hot solution was cooled to RT over weekend without stirring. After filtration, the cake was washed with MEK (20 mL twice) and vacuum dried at RT for 1 hour to afford light pink crystalline (6.185 g).

### Preparation of solid Form 1 (Step 2)

Free base DMSO solvate of the compound of Formula (I) obtained from Step 1 (150 g containing 128.4 g active compound) and 3852 mL of MEK (30 volume to the active compound) were charged to the reactor sequentially. The suspension was stirred at 150 rpm and heated to 79-80° C. at 4° C./min. A mixture of 20 mL of MEK and 19.3 mL of MSA (28.54 g, 1.1 eq. to the active compound) was added to the free base suspension at an addition rate of 1 mL/min while keeping the suspension with gentle reflux. After rinsing the MSA container using 5 mL of MEK, the solution was also added to the suspension at an addition rate of 1 mL/min. The suspension was stirred at 79-80° C. for additional 60 min, cooled to room temperature (21° C.) at 4° C./min. The suspension was filtered; the cake was washed with 500 mL of MEK twice and dried under suction for 20 min. The final compound (153.2 g) was obtained after further drying the cake at 35° C. for 4 hours under vacuum with N₂ stream.

Alternative 1:

Free base DMSO solvate of the compound of Formula (I) obtained from Step 1 (6 g containing 5.041 g active compound) and 200 mL of MEK (40 volume to the active compound) was charged to the reactor sequentially. The suspension was stirred at 250 rpm and heated to 79-80° C. at 4° C./min. Neat MSA (1121 mg, 1.1 eq. to the active compound) was added to the free base suspension drop-wise. After rinsing the MSA container using 1 mL of MEK, the solution was also added to the suspension drop-wise. The suspension was stirred at 79-80° C. for additional 120 min, then cooled to RT (21° C.) at 1.45° C./min. The suspension was then filtered; the cake was washed with 20 mL of MEK twice and dried under suction for 20 min. The final compound (6.03 g) was obtained after further drying the cake at 40° C. for 2 hours under vacuum with N₂ stream.

### Example 3

### Characterization of the Solid Form 1 of the Compound of Formula (I)

The XRPD pattern of the solid Form 1 of the compound of Formula (I) was collected on a Bruker D8 diffractometer using Cu Kα radiation (40 kV, 40 mA), 0-20 goniometer, and divergence of V4 and receiving slits, a Ge monochromator and a Lynxeye detector. The instrument was performance checked using a certified Corundum standard (NIST 1976). The software used for data collection was Diffrac Plus XRD Commander v2.5.0 and the data were analysed and presented using Diffrac Plus EVA v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection are: Angular range: 2 to 42° 20; Step size: 0.05° 20; Collection time: 0.5 s/step. The solid Form 1 of the compound of Formula (I) is characterized by the XRPD pattern of FIG. 2.

The compound of Formula (I) is further characterized by the ¹H-NMR chemical shift of FIG. 3. ¹H-NMR spectra were collected on a Bruker 400 MHz instrument equipped with an auto-sampler and controlled by a DRX400 console. Automated experiments were acquired using ICON-NMR v4.0.4 running with Topspin v1.3 using the standard Bruker loaded experiments. For non-routine spectroscopy, data were acquired through the use of Topspin alone. Samples were prepared in DMSO-d₆. Off-line analysis was carried out using ACD SpecManager v12.00.

### Example 4

### Measure Kinetic Solubility of Form 1 of the Compound of Formula (I)

About 10 mg of Form 1 of the compound of Formula (I) was added to 100 uL of either DMF or DMA at room temperature and stirred overnight, then the suspension was heated to 80° C. and the concentration of sample in either solvents was measured by HPLC at prior to heating, 0.5, 1, 2 and 4 hours post heating. The data are summarized in the Table 5a.

TABLE 5a

| | In DMF | In DMA |
|---|---|---|
| Prior to heating | 18.2 mg/mL | 33.0 mg/mL |
| 0.5 hour post heating | 42.0 mg/mL | 69.2 mg/mL |
| 1 hour post heating | 43.1 mg/mL | 68.0 mg/mL |
| 4 hours post heating | 45.5 mg/mL | 66.1 mg/mL |

SGF Experimental Details:

5 mg/mL stock solution made with pre-heated SGF at 37° C. Sample was vortexed before being centrifuged for 3 minutes at 13,000 rpm. Supernatant used for HPLC analysis of solubility and purity at T=0, 30 min, 1 h, 2 h and 4 h. If precipitation occurred the sample was centrifuged as above before analysis of the sample. Analysis performed in duplicate. Auto sampler set at 37° C.; pH of sample solutions taken after HPLC analysis at each time point (FIGS. 4A-4D).

SGF Spiked SIF Experimental Details:

5 mg/mL stock solution prepared in preheated SGF at 37° C. and shaken at 37° C. for 1 hour. After 1 hour the sample was centrifuged for 3 minutes at 13,000 rpm. The supernatant was used to spike preheated SIF (37° C.) at 5, 10 and 20 time dilutions. The concentration and purity was determined by HPLC analysis for each dilution level at T=0, 1 h, 2 h, 4 h, 6 h and 8 h. If precipitation occurred the sample was centrifuged as above before analysis of the sample. Analysis performed in duplicate. Auto sampler set at 37° C.; pH of sample solutions taken after HPLC analysis at each time point (FIGS. 4A-4D).

### Example 5

### Stability of Form 1 of the Compound of Formula (I)

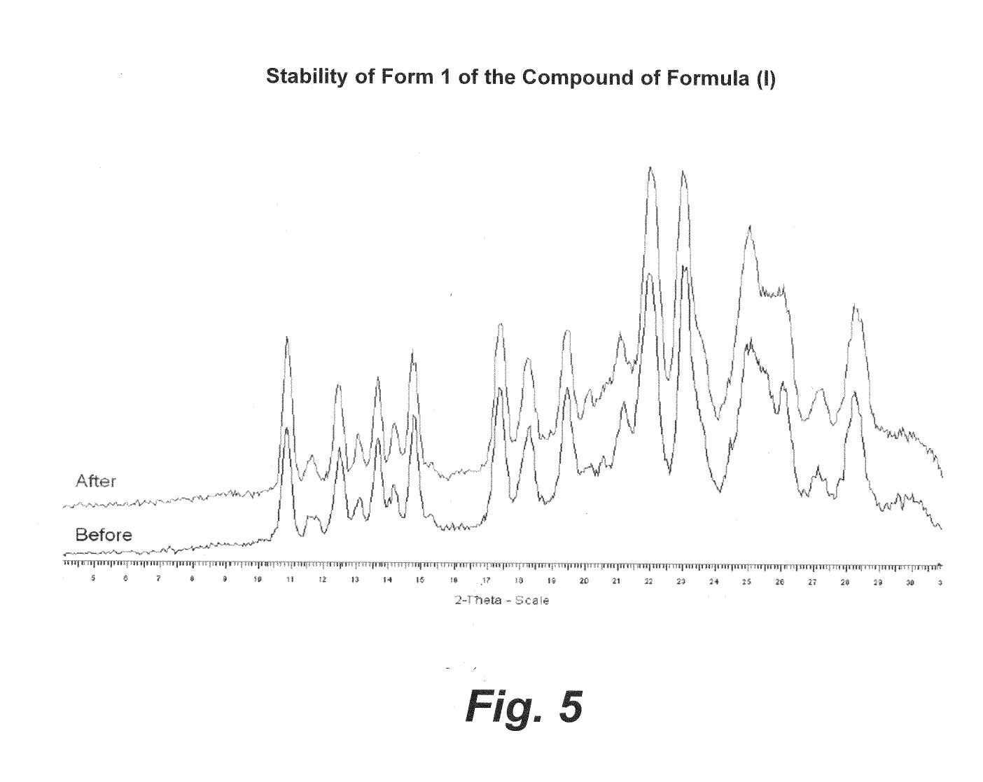

The stability of solid Form 1 of the compound of Formula (I) was determined by storing a sample in ambient conditions (25° C./40% RH) for 7 days followed by reanalysis with XRPD. Solid Form 1 of the compound of Formula (I) is stable under these conditions evidenced by no change in the XRPD pattern (FIG. 5).

### Example 6

### Measuring In Vivo Oral Exposure of the Mesylate Salt Form 1a Compound of Formula (I)

Solid Form 1 of the compound of Formula (I) also exhibits superior oral exposure. HCl salt powder comparator compound was made according to Example 9f (FIG. 6, Samples A and B). The API ethane sulfonate salt powder of the comparator compound was made according to Example 9g (FIG. 6, Sample D). The mesylate salt of the liquid comparator compound was made according to Example 9h (FIG. 6, Sample E). Compound of Formula (I) Form 1 salt powder was made according to Example 2 (FIG. 6, Sample C). Compound of Formula (I) test articles A, B, C and D were orally-administered as capsules (2 capsules at 500 mg/dose/dog) to fasted non-naive female beagle dogs (n=3 dogs/dose group). A flush of deionized (DI) water was administered immediately following capsule (5 mL) administration. In a separate study, test article E was prepared by accurately weighing out 200 mgs (±3%) compound of Formula (I) mesylate salt Form 1 (potency correction factor of 812 μg/mg) in a size 00 hard gelatin capsule, and stored refrigerated until time of use. Formulation E was orally-administered as a capsule (2 capsules at 400 mg/dose/dog) to fasted, non-naive male beagle dogs (n=3 dogs/dose group). A flush of deionized (DI) water was administered immediately following capsule (10 mL) administration.

Blood samples for all dose groups were collected at pre-dose, 0.25, 0.5, 1, 2, 4, 8, 12, and 24 hours post-dose from the cephalic vein and placed in K₂EDTA tubes and stored on wet ice, then centrifuged (3500 rpm for 10 minutes at 5° C.). Plasma samples were analyzed using a liquid chromatography-tandem mass spectrometry (LC/MS/MS) method with a lower limit of quantitation (LLOQ) of 0.005 μg/mL. Calibration standard concentrations over the range of 0.005 to 5.00 μg/mL were analyzed with a linear regression (1/x²) fitting method.

FIG. 6 shows the oral exposure of Compound of Formula (I) Form 1 mesylate salt as compared to the Compound of Formula (I) comparator compounds. The solid center lines of the box plot (112, 124, 134, 144, 154) represent the mean oral exposure expressed in Cmax/dose (for samples A, B, C, D, and E respectively). The horizontal dashed lines (114, 122, 132, 142, and 152) represent the median oral exposure expressed in Cmax/dose (for samples A, B, C, D, and E respectively). The tops and bottoms of the boxes represent the range of the data (114 and 110 sample A; 120 and 126 for sample B; 130 and 136 for sample C; 140 and 146 for sample D; 150 and 156 for sample E). The compound of Formula (I) Form 1 mesylate salt (Sample C) had higher oral exposure than any of the comparator compounds (FIG. 6).

### Example 7

### Formation of the Solid Form 4 of a Compound of Formula (I)

The free base of the compound of Formula (I) (8.0 g) was suspended in 20 volumes (160 mL) of MEK (HPLC grade ≧99.7%) and heated to 50° C. Neat methanesulfonic acid (1.2 mL, 1.1 eq.) diluted in MEK (HPLC grade ≧99.7% 4 vol, 32 mL) and added over 10 s to the suspension of free base. The suspensions were then cooled down to 25° C. at 1° C./min. The suspension was then filtered, washed with 3×40 mL of MEK and dried under suction for 5 minutes. The solid was then placed into the vacuum oven (˜5 mmHg) at room temperature for 72 hours. The sample was analyzed by XRPD (FIG. 7).

### Example 8

### Characterization of the Solid Form 4 of the Compound of Formula (I)

The XRPD pattern of the solid Form 4 of the compound of Formula (I) was collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Gobel multilayer mirror coupled with a pinhole collimator of 0.3 mm. A weekly performance check is carried out using a certified standard NIST 1976 Corundum (flat plate).

The beam divergence, i.e. the effective size of the X-ray beam on the sample, was approximately 4 mm. A θ-θ continuous scan mode was employed with a sample-detector distance of 20 cm which gives an effective 2θ range of 3.2°-29.7°. Typically the sample would be exposed to the X-ray beam for 120 seconds. The software used for data collection was GADDS for WNT 4.1.16 and the data were analysed and presented using Diffrac Plus EVA v15.0.0.0.

Ambient Conditions:

Samples run under ambient conditions were prepared as flat plate specimens using powder as received without grinding. Approximately 1-2 mg of the sample was lightly pressed on a glass slide to obtain a flat surface. Non-ambient conditions: Samples run under non-ambient conditions were mounted on a silicon wafer with heat-conducting compound. The sample was then heated to the appropriate temperature at 10° C./min and subsequently held isothermally for 1 minute before data collection was initiated.

Alternatively, X-Ray Powder Diffraction patterns were collected on a Bruker D8 diffractometer using Cu Kα radiation (40 kV, 40 mA), θ-2θ goniometer, and divergence of V4 and receiving slits, a Ge monochromator and a Lynxeye detector. The instrument was performance checked using a certified Corundum standard (NIST 1976). The software used for data collection was Diffrac Plus XRD Commander v2.5.0 and the data were analysed and presented using Diffrac Plus EVA v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection are: Angular range: 2 to 42° 2θ; Step size: 0.05° 2θ; Collection time: 0.5 s/step. The solid Form 4 of the compound of Formula (I) is characterized by the XRPD pattern of FIG. 7.

### Example 9

### Methods of Making Solid Forms of a Compound of Formula (I) Including the Solid Form 1 of Example 2, the Solid Form 4 of Example 3, Thirteen Other Solid Forms and Comparator Compounds

The solid Form 1 of the compound of Formula (I) is preferably made by the process of Example 2 which involves a two step process the first of which is a DMSO hot filtration solution crystallization step and the second of which is a slurry-to-slurry crystallization step. The solid Form 1 of the compound of Formula (I) can also be made by the methods of Examples 9b, 9c, 9d, and 9e. The solid Form 4 of the compound of Formula (I) is preferably made by the process of Example 7. The solid Form 4 of the compound of Formula (I) can also be made by the methods of Example 9b and 9d. Thirteen other solid forms of the compound of Formula (I) can be made according to the methods of Examples 9b, 9c, and 9d. All of the solid forms are characterized by XRPD patterns. The peaks for each of the solid forms greater than 30% intensity are listed in Table 6.

TABLE 6

| ID # | Form | XRPD Peaks (2Θ ± 0.3°) |
|---|---|---|
| 1 | Form 1 | 11.0, 12.5, 13.7, 14.8, 17.6, 22.3, 23.2 26.0, 28.7 |
| 2 | Form 2 | 4.8, 15.8, 17.8, 18.7, 21.2, 23.6, 24.1 |
| 3 | Form 3 | 11.4, 14.9, 15.4, 16.7, 18.5, 19.6, 20.5, 21.4, 21.7, |
| | | 22.5, 22.8, 23.9, 25.5, 25.8, 26.8, 27.9 |
| 4 | Form 4 | 10.9, 12.4, 13.7, 14.8, 17.5, 19.5, 22.3, 23.2, 25.2, |
| | | 25.4, 25.9, 26.4, |
| 5 | Form 5 | 5.6, 11.0, 18.9, 20.2, 22.0, 22.7, 26.1, 27.3 |
| 6 | Form 6 | 12.6, 18.5, 19.5, 20.7, 21.2, 21.6, 22.1, 23.1, 23.8, |
| | | 24.4, 25.1, 25.4, 28.1 |
| 7 | Form 7 | 12.6, 18.5, 19.5, 20.7, 21.6, 22.1, 23.1, 23.8, 24.4, |
| | | 25.1, 25.4, 28.1, 21.2 |
| 8 | Hydrate 1 | 10.4, 11.0, 19.6, 20.1, 22.4, 23.4 |
| 9 | Hydrate 2 | 12.5, 19.8, 21.5, 24.2, 27.6 |
| 10 | Hydrate 3 | 7.1, 10.8, 20.1, 21.7, 25.4, 25.7, 28.0 |
| 11 | Hydrate 4 | 6.5, 6.9, 10.5, 10.8, 14.4, 16.1, 18.3, 20.1, 20.8, |
| | | 21.3, 22.2, 22.5, 23.5, 24.2, 25.3 |
| 12 | Solvate 1 | 10.6, 11.5, 15.1, 15.7, 20.1, 21.0, 24.0, 24.8, 27.1 |
| 13 | Solvate 2 | 4.3, 14.0, 17.6, 17.9, 19.6, 21.1, 22.0, 22.9, 23.5, |
| | | 24.8, 25.5, 25.6, 26.4 |
| 14 | Solvate 3 | 10.8, 20.4, 22.6, 23.0, 23.4, 25.1, 25.5, 26.7, 28.1 |
| 15 | Solvate 4 | 4.5 |

### Example 9a

### Preparation of Amorphous Compound of Formula (I)

Seven different procedures were used to obtain amorphous mesylate salt of the compound of Formula (I). The solid obtained by each procedure was characterized by XRPD and ¹H-NMR. The amorphous material made by procedure 9a(7c) was used to make the solid forms disclosed in Example 9b.

Procedure 9a(1)—

The compound of Formula (I) (100 mg) was dissolved in DCM/EtOH 9:1 (2 mL) at room temperature. The solvent was evaporated under vacuum. A yellowish powder was obtained.

Procedure 9a(2)—

The compound of Formula (I) (100 mg) was dissolved in DCM/EtOH 9:1 (1.5 mL) at room temperature. The solution was poured into water (15 mL) at room temperature. A white powder precipitated out of the biphasic system. The solid was filtered and dried.

Procedure 9a(3)—

The compound of Formula (I) (100 mg) was dissolved in DCM/EtOH 9:1 (1.5 mL). The solution was added in heptane (7 mL). A yellowish powder crashed-out of solution immediately. The solid was filtered.

Procedure 9a(4)—

The compound of Formula (I) (2.0 g) was dissolved in DCM/EtOH 9:1 (30 mL) at room temperature. The solution was added dropwise into n-heptane (140 mL). A yellowish powder immediately precipitated out of the solution. The solid was filtered and dried in the vacuum oven (40° C., ˜5 mm Hg) for 4 hours.

Procedure 9a(5)—

The compound of Formula (I) (1.8 g) was suspended in DCM/EtOH 9:1 (30 mL) at room temperature. After ten minutes, the cloudy solution became a suspension. The solid was filtered, air dried.

Procedure 9a(6)—

The compound of Formula (I) (2.0 g) was dissolved in DCM/EtOH 9:1 (30 mL) at room temperature. The solution was filtered and added dropwise into n-heptane (200 mL) at 40° C. A solid precipitated out and was filtered.

Procedure 9a(7)—

The compound of Formula (I) (0.5 g) was dissolved in DCM/EtOH 9:1 (10 mL). The clear solution was then filtered and poured into n-heptane at room temperature (a) or 40° C. (b). The procedure was repeated again and the unfiltered solution was poured into n-heptane at 40° C. (c). The resulting solid was filtered, air dried.

TABLE 7

| | Solid Form (based | Solid Form (based |
|---|---|---|
| Procedure | on XRPD analysis) | on ¹H-NMR) |
| 9a(1) | Partially crystalline | N/A |
| 9a(2) | Crystalline | Free base |
| 9a(3) | Amorphous | Mesylate salt |
| 9a(4) | Partially crystalline | N/A |
| 9a(5) | Solvate 1 (DCM) | Mesylate salt |
| 9a(6) | Partially crystalline | N/A |
| 9a(7a) | Mainly Amorphous | Mesylate salt |
| 9a(7b) | Amorphous | Mesylate salt |
| 9a(7c) | Amorphous | Mesylate salt |
| 9a(7c) | Amorphous | Mesylate salt |
| 9a(7c) | Amorphous | N/A |
| 9a(7c) | Amorphous | N/A |

### Example 9b

### Formation of the Salt Forms of the Compound of Formula (I)

The method of Example 9a Procedure 9a(7c) was used to obtain the amorphous mesylate salt of the compound of Formula (I) used as the starting compound for the four procedures disclosed in Example 4d. Example 9b Procedure 9b(1) makes compounds number 1, 2, 9, 10, 12 disclosed in Table 6 depending on which solvent is selected (Table 8) Example 4d Procedure 9b(2) makes compounds number 1, 2, 9, 13, 14 disclosed in Table 6 depending on which solvent is selected (Table 9). Example 9b Procedure 9b(3) makes compounds number 2, 8, 9, 10, 11 disclosed in Table 6 depending on which solvent is selected (Table 10). Example 9b Procedure 9b(4) makes compounds number 3, 4, 5, 6, 7 disclosed in Table 6 depending on which initial staring form is selected (Table 11).

Procedure 9b(1)—

Amorphous mesylate salt of the compound of Formula (I) (40 mg) was suspended in different solvent systems (0.5 mL) and shaken at room temperature during 24 h. The resulting solids were filtered, air dried and analyzed by XRPD and ¹H NMR (Table 8).

TABLE 8

| Compound | | Solid Form (based | Stoichi- |
|---|---|---|---|
| ID # | Solvent | on XRPD analysis) | ometry |
| Comparative | n-Heptane | Amorphous | N/A |
| Example | | | |
| 2 | 1,4-Dioxane | Form 2 | N/A |
| Comparative | Toluene | mixture | N/A |
| Example | | | |
| 9 | Cumene | Hydrate 2 | N/A |
| 9 | TBME | Hydrate 2 | N/A |
| Comparative | Tetralin | Mixture | N/A |
| Example | | | |
| 9 | DIPE | Hydrate 2 | N/A |
| Comparative | Anisole | mixture | N/A |
| Example | | | |
| Comparative | Water | Free base | free base |
| Example | | | (NMR) |
| 9 | Ethyl Acetate | Hydrate 2 | N/A |
| 9 | Isopropyl Acetate | Hydrate 2 | N/A |
| 2 | IPA | Form 2 | 1:1 (NMR) |
| 1 | DME | Form 1 | 1:1 (NMR) |
| 2 | THF | Form 2 | N/A |
| 12 | DCM | Solvate 1 (DCM) | 1:1 (NMR) |
| 9 | MIBK | Hydrate 2 | N/A |
| 9 | MEK | Hydrate 2 | 1:1 (NMR) |
| 2 | Acetone | Form 2 | N/A |
| Comparative | Ethanol | Low crystalline | N/A |
| Example | | Hydrate 3 | |
| 10 | Acetonitrile | Hydrate 3 | 1:1 (NMR) |
| Comparative | Nitromethane | mixture | 1:1 (NMR) |
| Example | | | |
| 8 | Acetone/water (95:5) | Hydrate 1 | N/A |
| Comparative | EtOH/water (95:5) | Free base + | 2:1 (NMR) |
| Example | | extra peaks | |
| 8 | THF/water (95:5) | Hydrate 1 | 1:1 (NMR) |

Procedure 9b(2)—

Free base of the compound of Formula (I) (50 mg) was suspended in different solvent systems (0.5 mL), treated with MsOH (7.5 μL) and shaken at room temperature during 24 h. The resulting solids were filtered, air dried and analyzed by XRPD and ¹H NMR (Table 9).

TABLE 9

| Compound | | Solid Form (based | Stoichi- |
|---|---|---|---|
| ID # | Solvent | on XRPD analysis) | ometry |
| Comparative | n-Heptane | Free base | N/A |
| Example | | | |
| Comparative | 1,4-Dioxane | N/A | N/A |
| Example | | | |
| Comparative | Toluene | Free base | N/A |
| Example | | | |
| Comparative | Cumene | Free base | N/A |
| Example | | | |
| Comparative | TBME | Free base | N/A |
| Example | | | |
| Comparative | Tetralin | Free base | N/A |
| Example | | | |
| Comparative | DIPE | Free base | N/A |
| Example | | | |
| 13 | Anisole | Solvate 2 | 1:1 (NMR) |
| | | (anisole) | |
| Comparative | Water | Free base | N/A |
| Example | | | |
| 1 | Ethyl Acetate | Form 1 | N/A |
| 1 | Isopropyl Acetate | Form 1 | N/A |
| 2 | IPA | Form 2 | N/A |
| 1 | DME | Form 1 | N/A |
| 2 | THF | Form 2 | N/A |
| Comparative | DCM | N/A | N/A |
| Example | | | |
| Comparative | MIBK | Partially | N/A |
| Example | | crystalline | |
| 2 | MEK | Form 2 | N/A |
| Comparative | Acetone | mixture | N/A |
| Example | | | |
| 14 | Ethanol | Solvate 3 | 1:1 (NMR) |
| | | (EtOH) | |
| 1 | Acetonitrile | Form 1 | 1:1 (NMR) |
| 1 | Nitromethane | Form 1 | N/A |
| Comparative | Acetone/water (95:5) | N/A | N/A |
| Example | | | |
| Comparative | EtOH/water (95:5) | Free base | N/A |
| Example | | | |
| 9 | THF/water (95:5) | Hydrate 2 | 1:1 (NMR) |

Procedure 9b(3)—

Amorphous compound of Formula (I) (40 mg) was suspended in different solvent systems (0.5 mL) and stirred at 5° C. during 24 h. The resulting solids were filtered, air dried and analyzed by XRPD and ¹H NMR (Table 10).

TABLE 10

| Compound | | Solid Form (based | Stoichi- |
|---|---|---|---|
| ID # | Solvent | on XRPD analysis) | ometry |
| Comparative | n-Heptane | Amorphous | N/A |
| Example | | | |
| 2 | 1,4-Dioxane | Form 2 | N/A |
| Comparative | Toluene | Hydrate 2 + | N/A |
| Example | | Hydrate 1 | |
| 8 | Cumene | Hydrate 1 | N/A |
| Comparative | TBME | Hydrate 1 + | N/A |
| Example | | Solvate 2 | |
| 8 | Tetralin | Hydrate 1 | N/A |
| 8 | DIPE | Hydrate 1 | N/A |
| 9 | Anisole | Hydrate 2 | N/A |
| Comparative | Water | Free base | free base |
| Example | | | (NMR) |
| 9 | Ethyl Acetate | Hydrate 2 | N/A |
| 9 | Isopropyl Acetate | Hydrate 2 | N/A |
| 2 | IPA | Form 2 | N/A |
| 9 | DME | Hydrate 2 | N/A |
| 2 | THF | Form 2 | N/A |
| Comparative | DCM | DCM solvate | N/A |
| Example | | | |
| 9 | MIBK | Hydrate 2 | N/A |
| 9 | MEK | Hydrate 2 | N/A |
| 2 | Acetone | Form 2 | N/A |
| 11 | Ethanol | Hydrate 4 | 1:1(NMR) |
| 10 | Acetonitrile | Hydrate 3 | N/A |
| 100 | Nitromethane | Hydrate 3 | N/A |
| 9 | Acetone/water (95:5) | Hydrate 2 | N/A |
| Comparative | EtOH/water (95:5) | N/A | N/A |
| Example | | | |
| 8 | THF/water (95:5) | Hydrate 1 | N/A |

Procedure 9b(4)—

Variable temperature X-ray experiments (VT-XRPD) were carried out in different crystalline forms identified in the experiments described above and in some cases, new crystalline forms were observed (Table 11).

TABLE 11

| Compound | Initial | Tempera- | Solid Form (based |
|---|---|---|---|
| ID # | Solid Form | ture | on XRPD analysis) |
| 3 | Hydrate 1 | 140° C. | Form 3 |
| 4 | Hydrate 1 | 225° C. | Form 4 |
| 5 | Form 2 | 190° C. | Form 5 |
| 6 | Hydrate 2 | 150° C. | Form 6 |
| 7 | Hydrate 3 | 140° C. | Form 7 |

### Example 9c

### Crystallization with DCM/IPA and DCM/EtOH

Some crystallization tests were carried out using DCM/IPA and DCM/EtOH as a solvent system, using n-heptane as anti-solvent. Example 9c Procedure 9c(1) makes compounds number 1, and 2 disclosed in Table 6 depending on the amount of n-heptane used as an anti-solvent (Table 12). Form 2 disclosed in Table 6 is also made by Procedure 9c(2), Procedure 9c(3a), Procedure 9c(3b), Procedure 9c(4), Procedure 9c(5a) (Table 13). Compound number 1 disclosed in Table 6 was also made by Procedure 9c(5b) by further processing the material resulting from Procedure 9c(5a) (Table 13).

Procedure 9c(1):

Compound of Formula (I) free base was suspended in different solvent systems at 50° C. and treated with methanesulfonic acid (2.4 eq). The crystallization was carried out by addition of n-heptane as anti-solvent. After the addition of anti-solvent, the system was allowed to cool down to room temperature and the samples were filtered, air dried. XRPD was used to identify the solid forms made (Table 12).

TABLE 12

| | | | | | | | Solid |
|---|---|---|---|---|---|---|---|
| | | | | | | | Form |
| | | | | | | | (based |
| | Free | | | | | | on |
| Compound | base | Solvent | MsOH | Heptane | Observation after | Yield | XRPD |
| ID # | (mg) | (2 mL) | (μL) | (mL) | 48 h at 25° C. | (%) | analysis) |
| 1 | 100 | DCM/IPA | 33 | 0.5 | Slurry | 65% | Form 1 |
| | | (80:20) | | | | | |
| 2 | 100 | DCM/IPA | 33 | 1 | Slurry | 84% | Form 2 |
| | | (80:20) | | | | | |
| 2 | 100 | DCM/IPA | 33 | 2 | Slurry | 90% | Form 2 |
| | | (80:20) | | | | | |
| Comparative | 200 | DCM/EtOH | 66 | 0.5 | Clear solution | N/A | N/A |
| Example | | (85:15) | | | | | |
| Comparative | 200 | DCM/EtOH | 66 | 1 | Sticky gel | N/A | N/A |
| Example | | (85:15) | | | | | |
| Comparative | 200 | DCM/EtOH | 66 | 2 | Sticky gel | N/A | N/A |
| Example | | (85:15) | | | | | |

Procedure 9c(2):

Compound of Formula (I) free base (999.5 mg) was suspended in DCM/IPA (80:20, 20 mL) at room temperature and treated with methanesulfonic acid (2.4 eq). The clear solution was heated to reflux and the solvent was distilled out. After evaporation of ˜8 mL of solvent, the solution was seeded with Form 1 of the compound of Formula (I). After evaporation of an extra ˜2 mL of solvent, the system became a thick paste. IPA (5 mL) was then added at 1 mL/min. The resulting solid was filtered, air dried, and analyzed using XRPD (Table 13).

Procedure 9c(3):

The compound of Formula (I) free base (1.005 g) was suspended in DCM/IPA (80:20, 20 mL) at room temperature and treated with methanesulfonic acid (2.4 eq). The clear solution was seeded with Form lof the compound of Formula (I). n-Heptane (10 mL) was then added at 0.5 mL/min. At the end of the addition, a sample was taken and analyzed by XRPD (a). The suspension was then seeded again and heated to 70° C. to eliminate the DCM. When the internal temperature reached 70° C., a sample was taken and analyzed by XRPD (b). The suspension was allowed to cool down to room temperature, filtered, dried in the vacuum oven (25° C., ˜5 mm Hg) for 2 h and analyzed by XRPD (c) (Table 13).

Procedure 9c(4):

The compound of Formula (I) free base (1.003 g) was suspended in DCM/IPA (80:20, 20 mL) at room temperature and treated with methanesulfonic acid (2.4 eq). The clear solution was seeded with Form 1 of the compound of Formula (I). n-Heptane (16 mL) was then added over a period of 5 min. The DCM was distilled out of the solution and the suspension was heated at 70° C. overnight. The solid was filtered, dried in the vacuum oven (25° C., ˜5 mm Hg) for 2 h and analyzed using XRPD (Table 13).

Procedure 9c(5):

The compound of Formula (I) free base (500 mg) was suspended in DCM/IPA (80:20, 10 mL) and treated with methanesulfonic acid (2.4 eq). The clear solution was heated to reflux and seeded with Form 1 of the compound of Formula (I). MEK (2.5 mL) was then added over a period of 30 min. A sample was extracted and analyzed by XRPD (a). More MEK (2.5 mL) was added over a period of 30 min. The slurry was stirred at 70° C. for 3 hours and then allowed to cool down to room temperature. The sample was filtered, air dried and analyzed by XRPD (b) (Table 13).

TABLE 13

| Compound | | Yield | Solid Form (based |
|---|---|---|---|
| ID # | Procedure | (%) | on XRPD analysis) |
| 2 | 9c (2) | 95.7% | Form 2 |
| 2 | 9c (3a) | N/A | Form 2 |
| 2 | 9c (3b) | N/A | Form 2 |
| Comparative | 9c (3c) | 98.4% | Mixture |
| Example | | | |
| 2 | 9c (4) | 89.1% | Form 2 |
| 2 | 9c (5a) | N/A | Form 2 |
| 1 | 9c (5b) | 82.5% | Form 1 |

### Example 9d

### Crystallization with DMSO

Solid forms of the compound of Formula (I) can also be made using DMSO as the solvent. Procedure 9d(1) results in compounds number 1 and 15 disclosed in Table 6 depending on the solvent used (Table 14). Procedure 9d(5) makes compound I and 15 disclosed in Table 6 depending on the solvent used (Table 15). Procedure 9d(3) and Procedure 9d(4) make compound 15 disclosed in Table 6 (Table 16). Procedure 9d(6) makes compound 4 disclosed in Table 6 (Table 16). Procedure 9d(7) makes only compound I disclosed in Table 6 (Table 17).

Procedure 9d(1):

The compound of Formula (I) free base (50 mg) was suspended in DMSO (100 μL) at room temperature and treated with methanesulfonic acid (2.2 eq). The clear solution was split in four parts and added drop-wise into different solvents systems. The resulting solids are analyzed using XRPD (Table 14).

TABLE 14

| Compound | Solvent | | Solid Form (based |
|---|---|---|---|
| ID # | (1 mL) | Observation | on XRPD analysis) |
| Comparative | TMBE | Solid stuck | N/A |
| Example | | in walls | |
| 1 | MEK | Slurry | Form 1 |
| 15 | THF | Slurry | Solvate 4 (DMSO) |
| Comparative | n-Heptane | Solid stuck | N/A |
| Example | | in walls | |

Procedure 9d(2):

The compound of Formula (I) free base (50 mg) was suspended in DMSO (100 μL) and treated with methanesulfonic acid (2.4 eq). The clear solution was added dropwise into MEK (1 mL) at 50° C. The resulting suspensions were stirred at this temperature overnight (16 h) and then allowed to cool down to room temperature. The solid was then filtered, air-dried and analyzed using XRPD (Table 16).

Procedure 9d(3):

The compound of Formula (I) free base (50 mg) was suspended in DMSO (100 μL) and treated with methanesulfonic acid (2.4 eq). The clear solution was added dropwise into THF (1 mL) at 50° C. The resulting suspensions were stirred at this temperature overnight (16 h) and then allowed to cool down to room temperature. The solid was then filtered, air-dried and analyzed using XRPD (Table 16).

Procedure 9d(4):

The compound of Formula (I) free base (250 mg) was suspended in DMSO (1.0 mL), treated with methanesulfonic acid (2.4 eq) at 70° C. and treated with MEK (10 mL) at 1 mL/min. The suspension was stirred at this temperature for 30 min, then cooled down to room temperature. The samples were then filtered, air-dried and analyzed using XRPD (Table 16).

Procedure 9d(5):

The compound of Formula (I) free base (150 mg) was suspended in DMSO (600 μL) and treated with methanesulfonic acid (2.4 eq) at 70° C. The resulting clear solution was split in 3 parts. An aliquot of this solution (200 μl) was added dropwise to different solvent systems at different temperatures and stirred at this temperature for 30 min. The samples were then filtered, air-dried and analyzed using XRPD (Table 15).

TABLE 15

| Compound | | Solid Form (based |
|---|---|---|
| ID # | Solvent | on XRPD analysis) |
| 15 | THF | Solvate 4 |
| 15 | MEK | Solvate 4 |
| 1 | MIBK | Form 1 |

Procedure 9d(6):

The compound of Formula (I) free base (5 g) was suspended in DMSO (20 mL) and treated with methanesulfonic acid (2.4 eq) at 70° C. The resulting solution was added dropwise to MIBK (100 mL) over a period of 5 min. The resultant suspension was stirred at 70° C. for 10 hours and then cooled down to 25° C. at 0.1° C./min. The solid was filtered, washed with MIBK (3×20 mL), dried in the vacuum oven for 2 hours (50° C., ˜5 mmHg) and analyzed using XRPD (Table 16).

TABLE 16

| Compound | | Solid Form (based |
|---|---|---|
| ID # | Procedure | on XRPD analysis) |
| Comparative | 4d(2) | Poorly crystalline |
| Example | | Form 1 |
| 15 | 4d(3) | Solvate 4 |
| 15 | 4d(4) | Solvate 4 |
| 4 | 4d(6) | Form 4 |

Procedure 9d(7):

The compound of Formula (I) free base was suspended in DMSO (4 volumes) and treated with different amounts of methanesulfonic acid at different temperatures. MIBK was added dropwise at different addition rates and hold for different periods of time before cooling down at different cooling rates. The resulting solid was analyzed using XRPD (Table 17).

TABLE 17

| | | | | | | | | Solid |
|---|---|---|---|---|---|---|---|---|
| | Conditions | | | | | | | Form |
| | Temp. of | | | | | Cooling | | (based on |
| Compound | MIBK | Time of | Volumes | MsOH | Hold | Rate | | XRPD |
| ID # | addition | addition | of MIBK | (eq) | Time | (° C./min) | Scale (g) | analysis) |
| 1 | 70° C. | 60 min | 20 | 2.4 | 9 h | 0.1 | 5 | Form 1 |
| 1 | 100° C. | 30 min | 16 | 1.1 | 30 min | 0.5 | 2 | Form 1 |
| 1 | 100° C. | 30 min | 16 | 1.6 | 30 min | 0.5 | 2 | Form 1 |
| 1 | 90° C. | 6 min | 16 | 1.2 | 5 min | 1 | 1 | Form 1 |
| 1 | 90° C. | 6 min | 16 | 1.2 | 5 min | 1 | 1 | Form 1 |
| 1 | 90° C. | 6 min | 16 | 1.2 | 5 min | 1 | 1 | Form 1 |
| 1 | 100° C. | 6 min | 16 | 1.2 | 5 min | 1 | 1 | Form 1 |

### Example 9e

### Crystallization with DMSO/MIBK

Solid forms of the compound of Formula (I) can also be made using DMSO/MIBK as the solvent. Procedure 9e(1) and Procedure 9e(2) make compound number 1 disclosed in Table 6 (Table 18 and Table 19)

Procedure 9e(1):

The compound of Formula (I) free base (1 g) was suspended in DMSO/MIBK and treated with different amounts of methanesulfonic acid at different temperatures. MIBK (16 volumes) was added at different rates. After the addition of anti-solvent was completed, the systems were held at the corresponding temperature for 5 minutes, then cooled down to room temperature at different cooling rates and analyzed using XRPD (Table 18).

TABLE 18

| | Conditions | | | | | Solid Form |
|---|---|---|---|---|---|---|
| | | Temp. of | | | | (based on |
| Compound | Initial | MIBK | Time of | | Cooling Rate | XRPD |
| ID # | Condition | addition | addition | MeOH (eq) | (° C./min) | analysis) |
| 1 | 4 volumes at | 90° C. | 30 min | 0.95 | 0.5 | Form 1 |
| | 90° C. | | | | | |
| | DMSO/MIBK | | | | | |
| | (80:20) | | | | | |
| 1 | 4 volumes at | 90° C. | 30 min | 1.00 | 0.5 | Form 1 |
| | 90° C. | | | | | |
| | DMSO/MIBK | | | | | |
| | (80:20) | | | | | |
| 1 | 4 volumes at | 90° C. | 30 min | 1.05 | 0.5 | Form 1 |
| | 90° C. | | | | | |
| | DMSO/MIBK | | | | | |
| | (80:20) | | | | | |
| 1 | 4 volumes at | 90° C. | 30 min | 1.10 | 0.5 | Form 1 |
| | 90° C. | | | | | |
| | DMSO/MIBK | | | | | |
| | (80:20) | | | | | |
| 1 | 2 volumes of | 95° C. | 10 min | 0.95 | 0.5 | Form 1 |
| | DMSO/MIBK | | | | | |
| | (90:20) + 600 μL | | | | | |
| | MIBK at | | | | | |
| | 95° C. | | | | | |
| 1 | 3 volumes of | 95° C. | 10 min | 1.00 | 0.5 | Form 1 |
| | DMSO/MIBK | | | | | |
| | (90:20) + 700 μL | | | | | |
| | MIBK at | | | | | |
| | 95° C. | | | | | |
| 1 | 4 volumes of | 95° C. | 12 min | 1.05 | 0.5 | Form 1 |
| | DMSO/MIBK | | | | | |
| | (90:20) + 400 μL | | | | | |
| | MIBK at | | | | | |
| | 95° C. | | | | | |
| 1 | 4 volumes of | 95° C. | 13 min | 1.10 | 0.5 | Form 1 |
| | DMSO/MIBK | | | | | |
| | (90:20) + 500 μL | | | | | |
| | MIBK at | | | | | |
| | 95° C. | | | | | |

Procedure 9e(2):

The compound of Formula (I) free base was suspended in DMSO/MIBK and treated with different amounts of methanesulfonic acid at different temperatures. MIBK was added at different addition rates. After the addition of anti-solvent was completed, the systems were held at the corresponding temperature for 5 minutes. The system was then cooled down to room temperature at different cooling rates. The solids were filtered, washed with MIBK (3×5 vol), placed into the vacuum oven (50° C., ˜5 mm Hg) for 24 hours and analyzed using XRPD (Table 19).

TABLE 19

| | Conditions | | | | | | | | |
|---|---|---|---|---|---|---|---|---|---|
| | | Temp. | | | | | | | Solid Form |
| | | of | | | | | Cooling | | (based on |
| Compound | Initial | MIBK | Time of | Final | MeOH | | rate | Scale | XRPD |
| ID # | condition | addition | addition | volumes | (eq) | Seeds | (° C./min) | (g) | analysis) |
| 1 | 4 volumes at | 85° C. | 20 min | 20 | 1.10 | Yes | 1 | 1 g | Form 1 |
| | 90° C. | | | | | | | | |
| Comparative | 4 volumes of | 95° C. | 37 min | 20 | 1.10 | no | 0.5 | 10 g | Form 1 + |
| Example | DMSO/MIBK | | | | | | | | Solvate 4 |
| | (80:20) + | | | | | | | | |
| | 5 mL MIBK + | | | | | | | | |
| | 2 mL | | | | | | | | |
| | DMSO at | | | | | | | | |
| | 95° C. | | | | | | | | |
| | 4.5 volumes | 95° C. | 90 min | 20 | 1.10 | Yes | 0.5 | 10 g | Form 1 + |
| | at 95° C. | | | | | | | | Solvate 4 |
| Comparative | 4.5 volumes | 85° C. | 10 min | 20 | 1.00 | Yes | 0.2 | 2 g | Form 1 + |
| Example | of | | | | | | | | Solvate 4 |
| | DMSO/MIBK | | | | | | | | |
| | (80:20) at | | | | | | | | |
| | 95° C. | | | | | | | | |
| 1 | 4.5 volumes | 90° C. | 8 min | 20 | 1.00 | No | 0.2 | 2 g | Form 1 |
| | of | | | | | | | | |
| | DMSO/MIBK | | | | | | | | |
| | (80:20) at | | | | | | | | |
| | 95° C. | | | | | | | | |
| 1 | 4.5 volumes | 95° C. | 10 min | 20 | 1.00 | Yes | 0.2 | 2 g | Form 1 |
| | of | | | | | | | | |
| | DMSO/MIBK | | | | | | | | |
| | (80:20) at | | | | | | | | |
| | 95° C. | | | | | | | | |
| 1 | 4 volumes of | 90° C. | 21 min | 20 | 1.00 | No | 0.1 | 4 g | Form 1 |
| | DMSO/MIBK | | | | | | | | |
| | (80:20) at | | | | | | | | |
| | 100° C. | | | | | | | | |
| 1 | 4 volumes of | 90° C. | 19 min | 20 | 1.00 | No | 0.5 | 4 g | Form 1 |
| | DMSO/MIBK | | | | | | | | |
| | (80:20) at | | | | | | | | |
| | 100° C. | | | | | | | | |
| Comparative | 4 volumes of | 90° C. | 18 min | 25 | 1.00 | No | 0.2 | 4 g | Form 1 |
| Example | DMSO/MIBK | | | | | | | | contaminated |
| | (80:20) at | | | | | | | | with Solvate 4 |
| | 100° C. | | | | | | | | |
| Comparative | 4.25 volumes | 90° C. | 18 min | 20 | 1.00 | Yes | 0.1 | 4 g | Form 1 + |
| Example | of | | | | | | | | Solvate 4 |
| | DMSO/MIBK | | | | | | | | |
| | (80:20) at | | | | | | | | |
| | 95° C. | | | | | | | | |
| 1 | 4.25 volumes | 90° C. | 21 min | 25 | 1.00 | Yes | 0.1 | 4 g | Form 1 |
| | of | | | | | | | | |
| | DMSO/MIBK | | | | | | | | |
| | (80:20) at | | | | | | | | |
| | 95° C. | | | | | | | | |
| 1 | 4.25 volumes | 90° C. | 25 min | 30 | 1.00 | yes | 0.1 | 4 g | Form 1 |
| | of | | | | | | | | |
| | DMSO/MIBK | | | | | | | | |
| | (80:20) at | | | | | | | | |
| | 95° C. | | | | | | | | |

### Example 9f

### Formation of the Comparator Compound HCl Salt of a Compound of Formula (I)

The free base of the compound of Formula (I) (˜150 mg per experiment) was suspended in MEK or IPAc (4.5 mL) and heated to 50° C. Each sample was treated with 1 eq of hydrochloric acid at 50° C. and the suspensions were shaken at this temperature overnight (˜16 h). The mixtures were then allowed to cool down to room temperature, and the residual solids were filtered, air-dried and analyzed by XRPD. The HCl salt form can be characterized by XRPD peaks greater than 30% intensity expressed in degrees 2-Theta at angles)(±0.3°) of about 5.7, 10.9, 12.7, 14.7, 15.8, 19.5, 20.6, 22.8.

### Example 9g

### Formation of the Comparator Compound Ethane Sulfate Salt of a Compound of Formula (I)

The free base of the compound of Formula (I) (7.284 g, 15.3 mmol) was suspended in MEK (240 mL, 33 vol) at 70° C. Ethane sulfonic acid (1.940 g, 1.15 eq.) was dissolved in MEK (50 mL) and added drop-wise over 1 hour to the free base suspension. The temperature was maintained at 70° C. After 19 h, the slurry was cooled to 10° C. The suspension was filtered, the cake was washed with 2×10 mL of MEK, dried under vacuum at RT overnight. The salt was stored at 40° C./75% RH over 2.5 days. The sample was then stored at 40° C./96% RH over 18 hours and analyzed by XRPD. The ethane sulfonate salt form can be characterized by XRPD peaks greater than 30% intensity expressed in degrees 2-Theta at angles)(±0.3°) of about 6.8, 10.1, 10.7, 12.4, 14.3, 16.1, 17.3, 18.8, 19.8, 21.2, 22.1, 24.1, 24.7.

### Example 9h

### Formation of the Comparator Compound Methane Sulfate Salt of a Compound of Formula (I)

The comparator methane sulfonate salt of a compound of Formula (I) can be formed via a procedure analogous to that in Example 9g, wherein methane sulfonic acid replaces ethane sulfonic acid.

### Example 10

### Pharmaceutical Composition Containing the Compound of Formula (I)

The components of a pharmaceutically acceptable formulation can include a compound of a mesylate salt form of a compound of Formula (I) as the active ingredient. The formulated solid drug product unit dose can comprise the mesylate salt of the compound of Formula (I).

The pharmaceutical composition can be a unit dose ranging from 200 to 500 mg in a size “00” capsule or equivalent tablet size. If 500 mg active/unit dose is achieved then development for that technology will be targeted to the highest achievable dose.

Preferably, the pharmaceutical compositions comprising the compound of Formula (I) can be formulated to provide a reduction in pain following surgery (e.g., management of pain following surgery compared to placebo to achieve about 50-100% reduction in opiate use within the first 24 hours after surgery). The pharmaceutical compositions comprising the compound of Formula (I) can be indicated for use as an analgesic and/or anti-inflammatory therapeutic (e.g., to block acute pain and prevent or reduce inflammation at a wound site and prevent central sensitization). In one embodiment, the pharmaceutical compositions comprising the compound of Formula (I) can be administered BID for a suitable time period (e.g., 7-14 days) and provide analgesia within about 30 minutes of administration. Preferably, the pharmaceutical composition(s) comprising the compound of Formula (I) can provide clinically measureable decreases in pain scores, without respiratory depression and/or drug-induced CNS effects.

### Example 11

### Measuring In Vitro Inhibition of TRPA1

The in vitro inhibition of TRPA1 of the compound of Formula (I) was tested using the procedure outlined in del Camino et al., J. Neurosci., 30(45):15165-15174, incorporated herein by reference and described below. Data for TRPA1 inhibition and the selectivity of TRPA1 inhibition was obtained by this method for the compound of Formula (I) and included in Table 1 and Table 2. All currents were recorded in whole-cell configuration using EPC-9 and EPC-10 amplifiers and Patchmaster software (HEKA). Patch pipettes had a resistance of 1.5-3 MS2 and 60-75% of the series resistance was compensated. The standard pipette solution consisted of 140 mM CsAsp, 10 mM EGTA, 10 mM HEPES, 2.27 mM MgCl₂, 1.91 mM CaCl₂, 4 mM MgATP, and 0.1-0.3 mM Na₂GTP, with pH adjusted to 7.2 with CsOH. In addition, a solution containing 145 mM CsCl, 10 mM HEPES, 10 mM EGTA and 1 mM MgCl₂ (pH 7.2 adjusted with csOH) can be used. The standard bath solution contained 150 mM NaCl, 10 mM HEPES, 10 mM glucose, 4.5 mM KCl, 1 mM EGTA, 3 mM MgCl₂, with pH adjusted to 7.4 with NaOH. In some instances, 2 mM CaCl₂ was added in place of EGTA and the concentration of MgCl₂ was reduced to 1 mM.

Data were collected either by continuous recordings at −60 mV or by applying voltage ramps from a holding potential of 0 mV every 4 s. Continuous recordings were collected at 400 Hz and digitally filtered off-line at 10 Hz for presentation. Voltage ramps were applied from −100 mV to 100 mV over the course of 400 ms, and data were collected at 10 kHz and filtered at 2.9 kHz. Inward and outward currents were analyzed from the ramps at −80 and 80 mV, respectively. Liquid junction potential correction was not used.

Solutions were switched using a gravity-fed continuous focal perfusion system. To achieve rapid temperature changes, two temperature control and perfusion systems were employed simultaneously. For temperatures ≧22° C., a Warner Instruments bipolar temperature controller (TC-344B) and inline heater (SHM-8) were used. For temperatures below 22° C. a Warner Instruments temperature controller (CL-100) and thermal cooling module (TCM-1) were used. Temperatures were confirmed using a thermistor (Warner Instruments, TA-29), with temperatures at the recorded cell estimated to be within +/−2° C. of those reported.

IC₅₀ of compounds was estimated by testing each compound at 5 micromolar and 500 nanomolar. When 5 micromolar compound showed no block, IC₅₀ was estimated as >10 micromolar. When 5 micromolar compound showed 50% or less block, a rough estimate of IC₅₀ in the range of 5-10 micromolar could be made. IC₅₀ for compounds between 500 nanomolar and 5 micromolar was similarly estimated. Compounds blocking 50% or more at 500 nanomolar are retested at multiple concentrations, and the % block at each is fitted by standard equations to determine IC₅₀ accurately using a 5-6 point concentration/response experiment.

### Example 12

### Evaluation In Vivo Efficacy of the Compound of Formula (I)

### Example 12a

### Evaluating the In Vivo Efficacy of TRPA1 Inhibitor Compounds

The compound of Formula (I) was evaluated for activity in vivo. In some examples, comparator TRPA1 inhibitor compounds of Formula (II) or Formula (III) were also evaluated, as described in the examples below.

The comparator compound of Formula (II), and methods of making and using this compound are disclosed as the TRPA1 inhibitor compound 200 in U.S. Pat. No. 7,671,061 (filed Dec. 22, 2006, issued Mar. 2, 2010) and are incorporated herein by reference in their entirety.

The comparator compound of Formula (III) and methods of making and using this compound are described in FIG. 1C and are disclosed as the TRPA1 inhibitor compound of Formula (I) in PCT patent application PCT/US2009/069146 (published as WO2010/075353A1 on Jul. 1, 2010) and are incorporated herein by reference in their entirety.

The potency and pharmacokinetic (PK) properties of (a) the compound of Formula (I); and (b) comparator compound of Formula (III) were evaluated. Bioavailability was measured as well. A pharmacokinetic study was performed to obtain a plasma drug concentration vs. time plot for the drug after both intravenous (IV) and oral (PO) administration. The absolute bioavailability is the dose-corrected area under curve (AUC) non-intravenous divided by the dose-corrected AUC intravenous. The formula for calculating F for a drug administered by the oral route (PO) is given below.

The bioavailability was calculated using the equation shown below:

% F=AUC PO×Dose IV/AUC IV×Dose PO

### Human Plasma Protein Binding

The amount of compound in buffer (free fraction) and the amount of compound associated with the plasma fraction is determined by equilibrium dialysis; the amount of compound bound is expressed as a percentage. (Banker et al., Journal of Pharmaceutical Sciences (2003) 92(5): 967-74.)

In Table 20, an “A” indicates an IC₅₀ of less than 25 nanomolar; a “B” indicates an IC₅₀ of 25 nanomolar to less than 50 nanomolar; a “C” indicates an IC₅₀ of 50 nanomolar to less than 100 nanomolar; a “D” indicates an IC₅₀ of 100 nanomolar or greater.

The IC₅₀ for the compound of Formula (I), when tested against hTRPA1, was between 50 and 100 nanomolar. The compound of Formula (I) was less than 99% protein-bound and the bioavailability for fed rats was greater than 50%. The IC₅₀ for the compound of Formula (III), when tested against hTRPA1, was between 50 and 100 nanomolar. The compound of Formula (III) was greater than 99% protein-bound and the bioavailability for fed rats was between 1 and 25%.

TABLE 20

| Parameter | Formula (III) | Formula (I) |
|---|---|---|
| Potency (IC₅₀) | | |
| Human | A | C |
| Rat | C | D |
| Dog | A | D |
| Bioavailability (Rat) | | |
| Fed | Between 1 and 25% | Greater than 50% |
| Fasted | Between 25 and 50% | Between 25 and 50% |
| Human Plasma Protein | Greater than 99% | Less than 99% |
| Binding | | |

While the compound of Formula (III) was more potent in vitro, the compound of Formula (I) has in vivo properties that make it advantageous over the compound of Formula (III). As shown in the Table above, the compound of Formula (I) demonstrated less of a fed/fasted effect. Compounds with reduced fed/fasted effects in humans can lead to increased patient compliance. In addition, the compound of Formula (I) was less protein-bound than the compound of Formula (III). As a consequence, more of the compound is available to be distributed to the target tissues upon administration.

### Example 12b

### Formalin-Induced Pain Behavior In Vivo Rodent Model

The compound of Formula (I) and the comparator compound of Formula (II) were tested in the formalin-induced pain test reported by Dubuisson et al., Pain 1977 December; 4(2):161-74 (incorporated herein by reference in its entirety). Dubuisson et al. (1977) describe a method for assessing pain and analgesia in rats and cats. Briefly, dilute formalin (50 μL of 3% formalin) was injected into the plantar surface of the hind paw. The animal was promptly returned to an observation arena (standard Plexiglass rat cage), at which point a trained observer recorded the time the animal spent exhibiting pain behaviors (flinching, licking, biting of the injected paw/leg) for a period of 5 minutes. The individual responsible for counting the pain behaviors in a particular study was blinded to the treatment groups.

Rats were treated with the HCl salt of Compound (I) at various doses (3, 10, 30, and 50 mg/kg, IP) or with the vehicle (IP). The vehicle animals showed an average of about 85 seconds exhibiting pain behaviors (e.g., licking the paw). The treated animals showed an average of about 38 seconds exhibiting pain behaviors. Results are shown in FIG. 8 and Table 3.

### Example 12c

### Complete Freund's Adjuvant (CFA) Inflammatory In Vivo Rodent Pain Model

The compound of Formula (I), the comparator compound of Formula (II) and ketoprofen were tested by the CFA-induced pain test method reported in del Camino et al., J. Neurosci., 30(45):15165-15174, incorporated herein by reference in its entirety.

Briefly, the hind paw was sensitized to cold temperature (allodynic), by administering 0.1 mL of Complete Freund's Adjuvant (CFA) to the left hind paw. 2-3 days later, the time taken for the animal to lift its CFA-injected paw was recorded compared to its un-injected normal right hind paw. Animals were placed on the surface of the cold plate (1° C.) and the operator stopped testing at the instant when the animal displayed discomfort by flinching or lifting its paw from the plate (paw withdrawal latency, or PWL). To avoid tissue damage the maximum cut-off time was 5 minutes. Animals that were allodynic (average PWL to the first three pain behaviors <150 seconds for the CFA-injected hind paw: ˜≧50% difference between the normal and CFA-injected paw) were included in the study and subsequently randomized across treatment groups. The following day, the animals were dosed under blinded conditions. Following the 1-2 hour pre-treatment time, the post-dose PWL readings were again taken. The efficacy of the drug treatment was assessed by comparing the PWL in the drug treatment animals to those animals that receive the vehicle. Results are shown in FIG. 9.

### Example 12d

### Surgical Incision Pain Behavior In Vivo Rodent Model (FIG. 10)

The compound of Formula (I), the comparator compound of Formula (II) and ketoprofen were tested by the formalin-induced pain test method reported in Brennan et al., Pain, 1996 March; 64(3):493-501 incorporated herein by reference in its entirety. Briefly, in rats under anesthesia, a 1 cm incision through skin and underlying muscle was made in the bottom of one hind paw. The incision was sutured closed and the animals allowed to regain consciousness in their home cage before being placed on a special mesh rack. The blinded observer subjectively assessed and recorded each animal's pain score every 5 minutes for 1 hour. Pain scores were assigned as follows: Score of 0=Injured paw was held flat on the rack and was bearing weight (=uninjured paw); 1=Injured paw was slightly lifted from the rack but was bearing some weight; 2=Injured paw was flat but was bearing no weight, or heel was lifted high off the rack with only toes touching. At the end of each hour, pain scores were added up and the final score recorded (maximum score=39). In a typical study the efficacy of the drug treatment was determined by comparing the cumulative guarding scores at 1-2 and 3-4 hours following surgical injury to the cumulative guarding scores of animals that received the vehicle.

Sixty (60) mg/kg delivered intraperitoneally (2 doses of 30 mg/kg before and immediately after the surgery) reduced spontaneous pain for up to 4 hours after surgery, equivalent to ketoprofen (2 doses of 2 mg/kg intraperitoneally). Thirty (30) mg/kg compound of Formula (I) intraperitoneally (2 doses of 15 mg/kg before and immediately after the surgery) only reduced spontaneous pain for up to 2 hours after surgery.

### Example 13

### Toxicity Studies of the Compound of Formula (I)

### Example 13a

### Hepatotoxicity Serum Biomarker Study of the Compound of Formula (I) and a Comparator Compound of Formula (III)

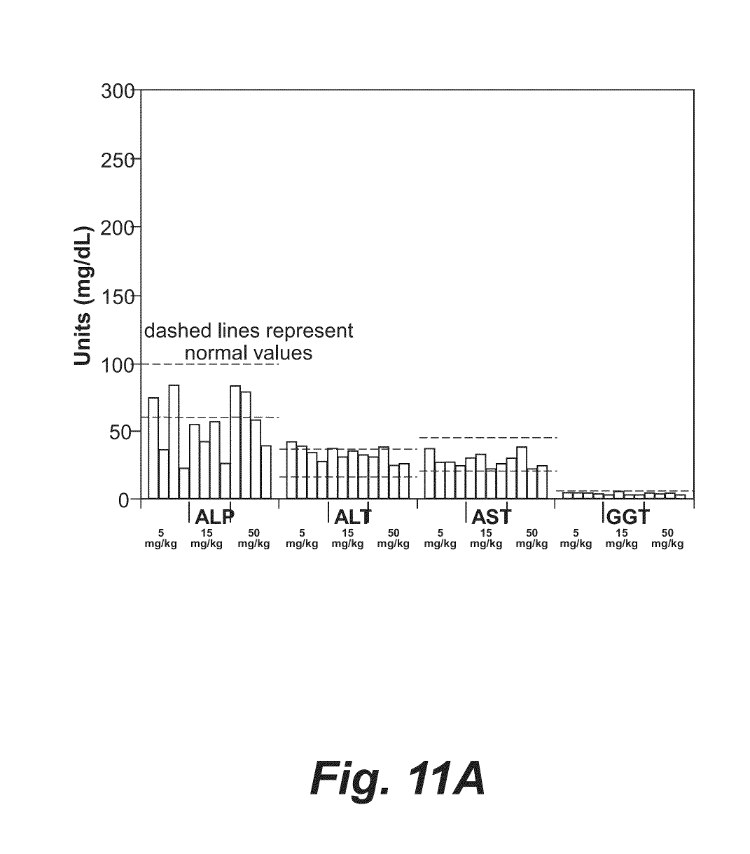

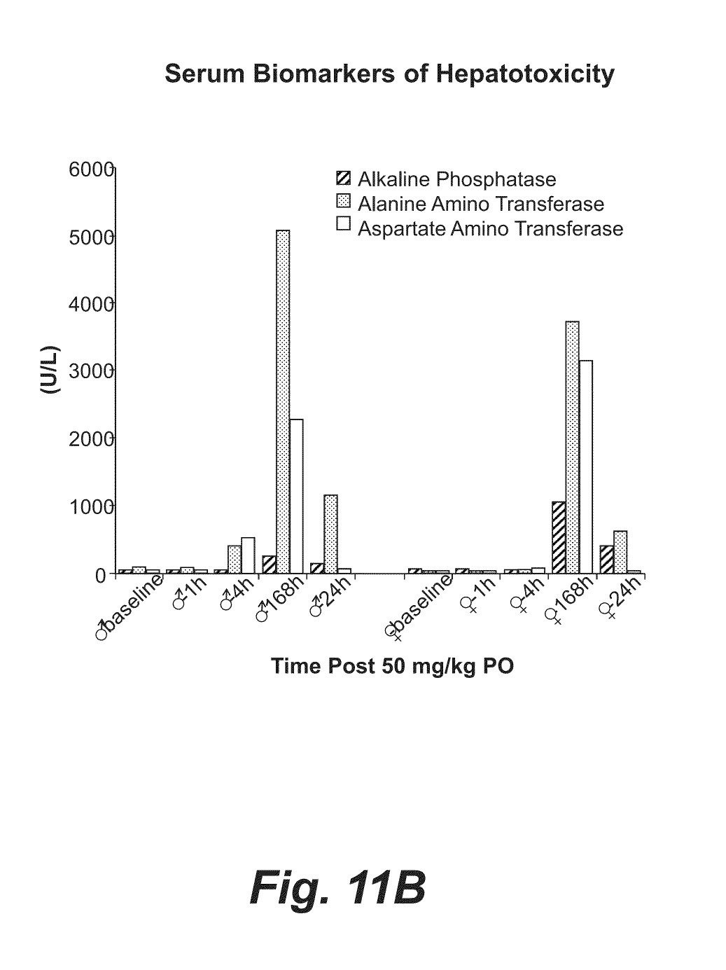

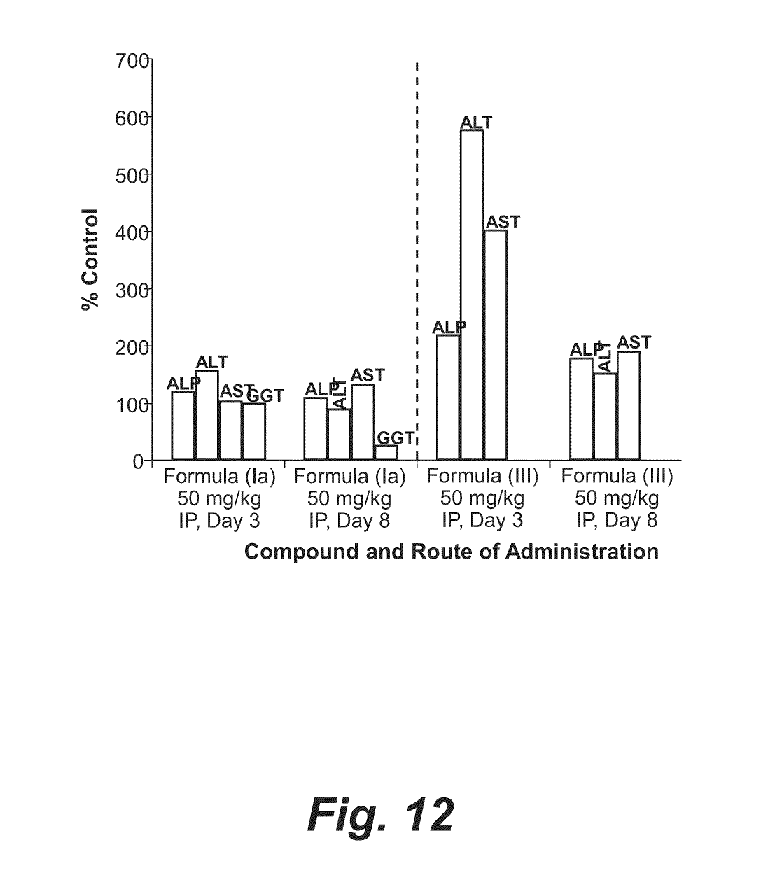

The compound of Formula (I) was orally dosed to female dogs at dose levels of 5, or 50 mg/kg using 30% Sulfobutylether β-cyclodextrin as the vehicle for assessment of safety as measured via serum chemistry biomarkers of hepatotoxicity or bile duct injury FIG. 11A, showing measurements of alanine aminotranferease [ALT], aspartate aminotranferease [AST], alkaline phosphatase [ALP] and gamma-glutamyl transferase [GGT] in the dogs at each dose level (each bar represents a measurement from 1 dog in the study). The data in FIG. 8A shows that the compound of Formula (I) did not elevate serum biomarkers of hepatotoxicity or acute phase response when dosed at 50 mg/kg PO (oral).

In contrast, the data in FIG. 11B shows that the comparator compound of Formula (III) did elevate serum biomarkers of hepatotoxicity. For example, the ALT levels were elevated up to about 60-fold in male dogs and up to about 130-fold in female beagle dogs following a single PO dose of 50 mg/kg.

### Example 13b

### Rodent Repeat Dose Toxicity Studies, Intraperitoneal (i.p.)

The compound of Formula (I) was evaluated in a 7-day repeat dose screening toxicity study in female rats. In order to maximize systemic exposure, rats were administered compound of Formula (I) i.p. at 50 mg/kg/day for 7 consecutive days, to obtain the results shown in FIG. 9. Clinical chemistry parameters were evaluated on Days 3 and 8. Histopathology was performed on select organs including the liver, kidney, spleen, and lung. After administration of the compound of Formula (I) at the 50 mg/kg IP dose, no adverse clinical signs, changes in body weight, or changes in clinical chemistry parameters were noted. No histopathological findings in the liver, kidney, spleen, or lung were observed after administration of the compound of Formula (I).

According to the pathologist's report, no adverse effects related to the compound of Formula (I) were identified in sections of liver harvested on study days 3 and 8 or spleen, kidney and lung harvested on study day 8.

In contrast, the data in FIG. 9 for compounds of Formula (III) shows that the comparator compound of Formula (III) did elevate serum biomarkers of hepatotoxicity as compared to Formula (I) following the 7-day repeat dose of 50 mg/kg/day for 7 consecutive days.

### Example 14

### Compositions Comprising the Mesylate Salt of Formula (I)

### Description of Composition of the Powder in Capsule (PIC) as a Solid Oral Drug Product (Mesylate Salt of the Compound of Formula (I))

### Dosage Form

The drug product, the compound of formula (I) mesylate salt has been developed as a capsule dosage form. The powder API is screened through a #20 mesh screen and filled into size 00 swedish orange hand gelatin capsule.

### Composition

The quantitative composition of the compound of formula (I) mesylate salt drug product is provided in Table 21. The drug product is provided as a 200 mg unit dose in size 00 swedish orange opaque hard gelatin capsules. The drug product consists of as is API in a hard gelatin capsule. The gelatin capsule and its components (i.e. red iron oxide, titanium dioxide, and gelatin) are USP/NF grade and are tested to confirm that the material meets current compendia standards (USP 32 NF 27) prior to use.

TABLE 21
The compound of formula (I) Mesylate
salt Drug Product Composition

| Component | Amount per unit | Function |
|---|---|---|
| the compound of formula (I) | 200 mg/capsule | Active |
| Mesylate salt (active) | | |
| FDA/E172 Red iron Oxide | 1.1817% of capsule | Capsule shell |
| | shell weight^{c} | colorant |
| Titanium Dioxide | 0.4916% of capsule | Capsule shell |
| | shell weight^{c} | opacifier |
| Gelatin | 98.3267% of capsule | Capsule shell |
| | shell weight^{c} | structure |

^{c}Average capsule weight is between 111 mg and 125 mg.

### Description of Composition of the Coated Tablet as a Solid Oral Drug Product (the Compound of Formula (I) Mesylate Salt)

There is a need for solid compositions of the mesylate salt of the compound of formula (I) with improved physical and chemical stability in the solid form (i.e., higher total percent the compound of formula (I) purity over time), providing advantages of improved dissolution and solubility, longer shelf life, increased tolerance for more varied storage conditions (e.g., higher temperature or humidity) and increased chemical stability. The present invention provides the compound of formula (I) mesylate salt compositions with improved the compound of formula (I) solubility profiles.

### Dosage Form

The drug product, the compound of formula (I) mesylate salt has been developed as a coated tablet dosage form. The powder API is screened through a #20 mesh screen and further blended with excipients including colloidal silicon dioxide, croscarmellose sodium, microcrystalline cellulose, lactose and magnesium stearate and or polyvinylpyrrolidone and then compressed into tablets using either direct compression or dry/wet granulation techniques. The tablet cores are coated with Opadry II 85F white.

### Composition

The quantitative composition of the compound of formula (I) mesylate salt drug product processed using either direct compression or dry/wet granulation techniques are provided in Table 22, 23 and 24 respectively. The drug product is provided as a 600 mg unit dose in 1000 mg size coated tablet. The drug product consists of API and excipients including colloidal silicon dioxide, croscarmellose sodium, microcrystalline cellulose, lactose, magnesium stearate and opadry II 85 F. The excipients are USP/NF grade and are tested to confirm that the material meets current compendia standards (USP 32 NF 27) prior to use.

TABLE 22
The compound of formula (I) mesylate salt Drug Product
Composition using direct compression technique

| | % | Amount per unit | |
|---|---|---|---|
| Component | Formula | tablet (mg) | Function |
| the compound of formula | 60 | 600 | Active |
| (I) mesylate salt (active) | | | |
| Microcrystalline cellulose; | 14 | 140 | Ductile filler |
| Avicel PH 101 | | | |
| Lactose monohydrate | 14 | 140 | Brittle filler |
| (310 grade) | | | |
| Croscarmellose sodium; | 10.0 | 100 | Disintegrant |
| Ac-Di-Sol | | | |
| Colloidal silicon dioxide; | 1.0 | 10 | Glidant |
| Cab-O-Sil M5P | | | |
| Magnesium stearate | 1.0 | 10 | Lubricant |
| Core Total | 100.0 | 1000.0 | |
| OPADRY 85 IIF white | 5 | 50 | Non-functional |
| | | | coating |

TABLE 23
The compound of formula (I) mesylate salt Drug Product
Composition using dry granulation technique

| | | | |
|---|---|---|---|
| | % | Amount per unit | |
| Component | Formula | tablet (mg) | Function |

Intra Granular

| | | | |
|---|---|---|---|
| the compound of formula | 60 | 600 | Active |
| (I) mesylate salt (active) | | | |
| Microcrystalline cellulose; | 6 | 60 | Ductile filler |
| Avicel PH 101 | | | |
| Lactose monohydrate | 24 | 240 | Brittle filler |
| (310 grade) | | | |
| Croscarmellose sodium; | 8.5 | 85 | Disintegrant |
| Ac-Di-Sol | | | |
| Colloidal silicon dioxide; | 0.25 | 2.5 | Glidant |
| Cab-O-Sil M5P | | | |
| Magnesium stearate | 0.5 | 5 | Lubricant |

Extra Granular

| | | | |
|---|---|---|---|
| Colloidal silicon dioxide; | 0.25 | 2.5 | Glidant |
| Cab-O-Sil M5P | | | |
| Magnesium stearate | 0.5 | 5 | Lubricant |
| Core Total | 100.0 | 1000.0 | |
| OPADRY 85 IIF white | 5 | 50 | Non-functional |
| | | | coating |

TABLE 24
The compound of formula (I) mesylate salt Drug Product
Composition using wet granulation technique

| | | | |
|---|---|---|---|
| | % | Amount per unit | |
| Component | Formula | tablet (mg) | Function |

Intra Granular

| | | | |
|---|---|---|---|
| the compound of formula | 60 | 600 | Active |
| (I) mesylate salt (active) | | | |
| Microcrystalline cellulose; | 6 | 60 | Ductile filler |
| Avicel PH 101 | | | |
| Lactose monohydrate | 24 | 240 | Brittle filler |
| (310 grade) | | | |
| Croscarmellose sodium; | 2 | 20 | Disintegrant |
| Ac-Di-Sol | | | |
| Polyvinylpyrrolidone; | 2 | 20 | Plasticizer, |
| PVP K 28/32; 10% binding | | | Binder |
| solution in water | | | |

Extra Granular

| | | | |
|---|---|---|---|
| Croscarmellose sodium; | 5 | 50 | Disintegrant |
| Ac-Di-Sol | | | |
| Colloidal silicon dioxide; | 0.5 | 5 | Glidant |
| Cab-O-Sil M5P | | | |
| Magnesium stearate | 0.5 | 5 | Lubricant |
| Core Total | 100.0 | 1000.0 | |
| OPADRY 85 IIF white | 5 | 50 | Non-functional |
| | | | coating |

### The Compound of Formula (I) Mesylate Salt Drug Product Composition Using Direct Compression Technique

Comparative formulation was prepared as a 600 mg coated tablet using API screening through #20 mesh sieve and blending of the compound of formula (I) with excipients followed by direct compression of the compound of formula (I). This comparative formulation incorporates direct compression using a suitable blender to mix API and excipients, which then feeds a chute to the tabletting process where it is sized and compressed into a tablet. The tablet core is the coated in a pan coater supplying opadry II 85F as a spray feed to achieve 5% weight gain.

### The Compound of Formula (I) Mesylate Salt Drug Product Composition Using Dry Granulation Technique

### Blending/Roller Compaction:

1. The API and Silicon Dioxide is charged to the V-Blender and blended for 5 minutes.
2. The resultant blend is passed through an Oscillating mill equipped with a 20 mesh screen.
3. The screened material is blended for 5 minutes.
4. An equal amount of blend from Step #3 is bag blended with Croscarmellose Sodium, Lactose monohydrate, microcrystalline cellulose and magnesium stearate. The blended material is passed through a #20 mesh screen and blended for 10 minutes.
5. The blended material from Step #4 is granulated using a roller compactor.
6. The roller compacted material from Step #5 is passed through an oscillating mill equipped with 20 mesh screen and transferred to the V-blender.
7. Extra-granular magnesium stearate is adjusted based upon the milled material from Step #6.
8. An equal volume of blend from Step #7 is removed and bag blended with the Magnesium

Stearate and screened through a 20 mesh hand screen. The material is added to the V-Blender and blended for 3 minutes.

### Compression:

The granulated material was compressed (manually) into 1000 mg tablets (weight of a 600 mg unit dose if the API potency is approximately 60.0% of blend). The blend is charged into the tablet press hopper and

compressed into tablets to a target weight of 1000.0 mg to a target hardness of 18-20 Kp.

### Coating:

A theoretical quantity of 100 g of a 20% suspension will be needed to apply coating to the tablet cores. The cores will be charged into the expansion chamber of a conventional pan coater and the prepared coating suspension will be used to achieve the 5% coat.

### The Compound of Formula (I) Mesylate Salt Drug Product Composition Using Wet Granulation Technique

### Blending/End Point Detection:

1. The API along with Croscarmellose Sodium, Lactose monohydrate and microcrystalline cellulose is screened through a 40 mesh screen.
2. The API is blended with Croscarmellose Sodium, Lactose monohydrate and microcrystalline cellulose for 5 minutes.
3. The blended material from Step #2 is granulated using a 10% PVP K28/32 solution in water using a high shear mixer until granulation end point is achieved.
4. The wet granulated material from Step #3 is passed through a mesh 20 screen and transferred to an oven for overnight drying at 35 degree Celcius.
5. Extra-granular Croscarmellose Sodium, colloidal silicon dioxide and magnesium stearate is adjusted based upon the milled material from Step #4.
6. An equal volume of blend from Step #4 is removed and blended with the Croscarmellose Sodium, colloidal silicon dioxide and magnesium stearate and screened through a 20 mesh hand screen. The material is added to the V-Blender and blended for 5 minutes.

### Compression:

The granulated material was compressed (manually) into 1000 mg tablets (weight of a 600 mg unit dose if the API potency is approximately 60.0% of blend). The blend is charged into the tablet press hopper and compressed into tablets to a target weight of 1000.0 mg to a target hardness of 18-20 Kp.

### Coating:

A theoretical quantity of 100 g of a 20% suspension will be needed to apply coating to the tablet cores. The cores will be charged into the expansion chamber of a conventional pan coater and the prepared coating suspension will be used to achieve the 5% coat.

### Example 15

### PK Comparison Studies on Various Salts of Formula (I)

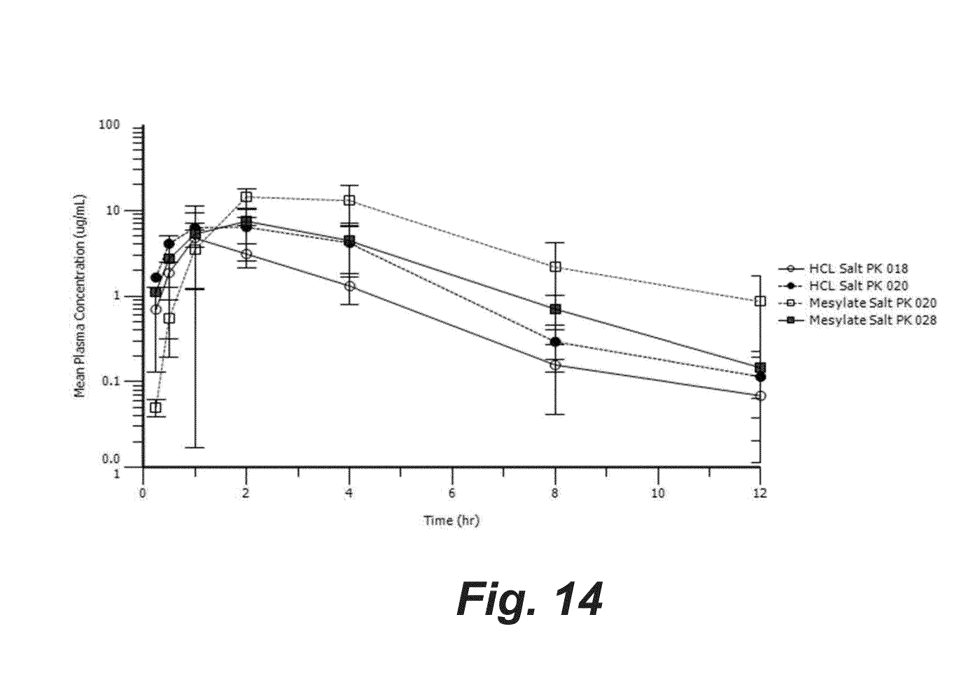

FIG. 14 shows PK studies in dogs comparing the plasma concentration over time of the mesylate salt of the compound of Formula (I) to the hydrochloride salt of the compound of Formula (I). These studies were based on administration of a 50 mg/Kg dose of the compound. As can be seen, the overall PK exposure was higher for the mesylate salt (“API in capsule”) as compared to the hydrochloride salt (“API in capsule”).

### Example 16

### Particle Size Preparation for Dry Powder for Inhalation

Described in this example are methods of making particles of the mesylate salt of the compound of Formula (I) useful for dry powder inhalation applications.

An exercise was conducted in order to identify which anti-solvents would be more suitable for the size reduction step of crystalline mesylate salt. Compound suspensions in different anti-solvents were prepared (all with 5% w/w solids content) and qualitatively evaluated. Water, absolute ethanol, n-heptane, acetone, ethyl acetate, and methylethylketone were tested. n-Heptane and ethyl acetate were selected as the most promising anti-solvent systems for the size reduction step, considering that (i) a very low amount of the mesylate salt was solubilized as determined by both the filtration test and HPLC (solubility in n-heptane seems to be the lowest), and (ii) the mesylate salt suspensions were relatively stable (although the ethyl acetate suspension was found to be more stable than the n-heptane suspension).

Size reduction and spray drying steps were conducted and high yields were obtained.

Fluid milling steps were then employed. The fluid milling technology enabled two distinct and well defined particle size distributions, namely with a Dv50 of 6.8 μm (span 1.9) and 2.7 μm (span 1.6), in order to deliver the mesylate salt via the inhalation route either for cough treatment (upper respiratory airways) or COPD (lower respiratory airways) treatment. The target particle sizes were achieved when using ethyl acetate as anti-solvent without any change on the compound's crystallinity or chemical purity, and with good process yields.

Aerodynamic characterization was also obtained. The aerodynamic properties of the fluid milled powders were initially assessed by gravimetric shot weight and Fast Screening Impactor (FSI), and further characterized in detail by Next Generation Impactor (NGI) without any further formulation work. The determined properties of the powders were consistent across the tests and adequate performance was obtained for both the manufactured fluid milled powders considering their target therapeutical application.

### Example 17

### Evaluation of Glass Transition Temperatures of Salt Forms

This experiment involves studies on the glass transition temperatures of the hydrochloride and mesylate salt forms of Formula (I). To predict long-term stability, mDSC was used to determine whether amorphous molecular dispersions had been formed and to measure the Tgs of the spray dried dispersions (SDDs). If SDDs are stored at or near their Tg, physical changes such as crystallization are possible. As a result, a high Tg is desirable.

In addition, samples were tested with and without exposure to high relative humidity (RH) to identify appropriate storage conditions for long-term stability and to assess the need for protective packaging.

HCl and mesylate salt SDDs were manufactured at 40% drug loading on a mini spray dryer for use in initial feasibility tests. The HCl salt formulation consisted of 53.82 mg compound, 71.18 mg hydroxypropyl methyl cellulose acetate succinate (HPMCAS), and 6.125 g methanol. The mesylate salt formulation consisted of 60.0 mg compound, 65.0 mg HPMCAS, and 6.125 g methanol. Secondary drying was performed to reduce the residual-solvent content of the SDDs by vacuum-drying them for 17 to 20 hours.

Samples of each formulation were exposed overnight to 75% relative humidity (RH) and the results were compared to those for samples maintained at less than <5% RH.

mDSC analysis showed that the mesylate salt SDDs were homogeneous amorphous molecular dispersions (characterized by a single Tg) when tested at <5% RH and 75% RH. As shown in Table 26, the mesylate salt SDDs have Tgs that are high enough to ensure long-term physical stability if they are protected from high humidity.

Likewise, mDSC analysis showed that the HCl salt SDDs were homogeneous amorphous molecular dispersions (as characterized by a single Tg) when tested at <5% RH. However, the HCl salt SDD samples were not tested after storage at 75% RH since overnight exposure to 75% RH resulted in discoloration. The formulations turned increasingly yellow as a function of drug loading.

TABLE 26
Tg for Wet and Dry SDD Samples

| Sample | Dry (<5% RH) | Wet (75% RH) |
|---|---|---|
| 40% mesylate salt SDD | 127 | 60 |
| 50% mesylate salt SDD | 123 | 61 |
| 60% mesylate salt SDD | 118 | 72 |
| 40% HCl salt SDD | 132 | Not tested |
| 50% HCl salt SDD | 129 | Not tested |
| 60% HCl salt SDD | 124 | Not tested |

### Example 18

### Tablet Formulation Through Spray Dried Dispersion (SDD)

Provided below is a method of preparing 22 tablets using a micronized form of the mesylate salt of Formula (I) for pharmacokinetic studies in dogs. (Formula was calculated for 25 tablets to account for any loss)

| | | | Quantity for |
|---|---|---|---|
| Ingredients | Function | % weight | 25 tabs (gms) |
| mesylate salt SDD powder | Active | 50 | 13.95 |
| | ingredient | | |

Intended Dose was 200 mgs; accounting for potency of the SDD powder
(360 μg/mg), weight of SDD powder required per tablet was found to be
558 mgs. Hence for 25 tablets the weight will be 25 × 0.558

| | | | |
|---|---|---|---|
| Lactose Monohydrate | Ductile | 25 | 6.975 |
| (310 NF grade) | Filler | | |
| Microcrystalline Cellulose | Binder | 7 | 1.953 |
| (Avicel PH101) | | | |
| Croscarmellose Sodium | Disintegrant | 8 | 2.232 |
| (Ac-di-sol) | | | |
| Colloidal Silicon Dioxide | Glidant | 0.5 | 0.14 |
| (Cab-o-sil) | | | |
| Magnesium Sterate | Lubricant | 0.5 | 0.14 |

Extragranular Ingredients

| | | | |
|---|---|---|---|
| Croscarmellose Sodium | Disintegrant | 8 | 2.232 |
| (Ac-di-sol) | | | |
| Colloidal Silicon Dioxide | Glidant | 0.5 | 0.14 |
| (Cab-o-sil) | | | |
| Magnesium Sterate | Lubricant | 0.5 | 0.14 |

The tablets in this experiment were prepared using a dry granulation method.

### Intra-Grnaular Powder Mixture:

1) SDD powder was weighed accurately in a labeled container and kept aside. The other intragranular ingredients were weighed accurately on different weigh papers.

2) Weighed amounts of active compound, Cab-o-sil and lactose were mixed well in a mortar and pestle. The other ingredients were then added and the intragranular mixture was poured into 250 mL plastic container and closed tightly.

3) The container was then placed in Turbula® mixer from GlenMills and mixed thoroughly for about 15 mins.

Slugging:

1) The homogenous mixture was then used to prepare lose slugs with hardness of about 1-2 kP.

2) A 1000 mg capacity Thomas Engineering single station tablet punch and dye were used to prepare slugs.

3) The pressure used to punch was about 500-600 psi.

Extra-Granular Powder Mixture:

1) All the slugs prepared were collected on a sieve USP standard sieve mesh #20 (mesh size 850μ).

2) The slugs were then broken and passed through this sieve collecting the granules on a collector pan.

3) After all the slugs were broken and passed through, granules were collected in another labeled 80 mL plastic container.

4) Extra-grnaular excipients were then accurately weighed and transferred to the labeled container

5) This container was placed in Turbula® mixer from GlenMills and mixed thoroughly for about 30 mins to ensure uniform mixture of the contents.

Tablet Compression:

1) 558 mgs of the final blend ready for compression was weighed accurately and added to a die with 500 mg capacity from Natoli engineering.

2) The powder blend was then compressed at a pressure of about 1200-1300 psi to give a final hardness of about 8-10 kP

3) The tablets punched were collected in a labeled container.

Analysis:

4) Hardness: The hardness, thickness and diameter of the tablets were tested using the CALEVA THT15 instrument. Two units were tested for each of the tests.

5) Disintegration: Disintegration tests were carried out using a USP standardized instrument, LIJ-2 from Vanguard Pharmaceutical Machinery, using a standard protocol. 0.01N HCl was used as a disintegration medium and 37° C. as the temperature during the test.

6) Dissolution: Distek's Evolution 6100 was used to perform in vitro release testing of the mesylate salt from the tablet. 0.1N HCl (pH 1.2) was used as the dissolution medium at 37° C. Two (2) tablets were tested.

| Test | Result obtained | |
|---|---|---|
| Hardness | 8 | kP |
| Disintegration | 3-5 | mins |

| | |
|---|---|
| Dissolution | 88% release after 45 mins |

### INCORPORATION BY REFERENCE

All publications and patents mentioned herein are hereby incorporated by reference in their entirety as if each individual publication or patent was specifically and individually indicated to be incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A crystalline mesylate salt of the compound of Formula (I): 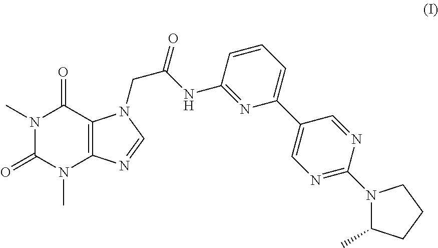 [SMILES: C[C@H]1CCCN1c1ncc(-c2cccc(NC(=O)Cn3cnc4c3c(=O)n(C)c(=O)n4C)n2)cn1]
wherein the crystalline mesylate salt is characterized by an X-ray powder diffraction pattern including peaks (2θ±0.3°) at 11.0, 12.5, 13.7, 14.8, 17.6, 22.3, 23.2 26.0, 28.7.

2. Use of a crystalline mesylate salt of the compound of Formula (I) in the manufacture of a pharmaceutical composition for treating a pain 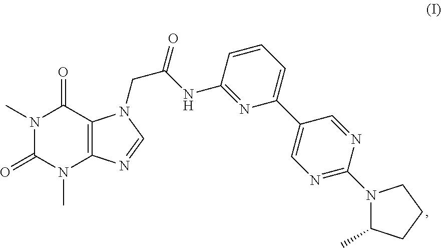 [SMILES: C[C@H]1CCCN1c1ncc(-c2cccc(NC(=O)Cn3cnc4c3c(=O)n(C)c(=O)n4C)n2)cn1]
wherein the crystalline mesylate salt is characterized by an X-ray powder diffraction pattern with peaks (2θ±0.3°) at 11.0, 12.5, 13.7, 14.8, 17.6, 22.3, 23.2 26.0, 28.7.

3. The use of claim 2, wherein the pharmaceutical composition is formulated for oral delivery.

4. The use of claim 3, wherein the pharmaceutical composition comprises a crystalline mesylate salt of the compound of Formula (I), wherein the crystalline mesylate salt is characterized by an X-ray powder diffraction pattern with peaks (2θ±0.3°) at 11.0, 12.5, 13.7, 14.8, 17.6, 22.3, 23.2 26.0, 28.7.

5. A pharmaceutical composition comprising a mesylate salt of the compound of Formula (I):

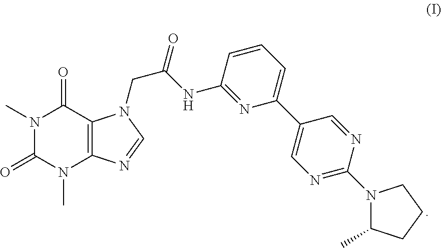 [SMILES: C[C@H]1CCCN1c1ncc(-c2cccc(NC(=O)Cn3cnc4c3c(=O)n(C)c(=O)n4C)n2)cn1]

6. The pharmaceutical composition of claim 5, further comprising a lactose, and one or more cellulose polymers.

7. The pharmaceutical composition of claim 6, wherein the mesylate salt is in crystalline form.

8. The pharmaceutical composition of claim 7, wherein the crystalline mesylate salt is characterized by an X-ray powder diffraction pattern with peaks (2θ±0.3°) at 11.0, 12.5, 13.7, 14.8, 17.6, 22.3, 23.2 26.0, 28.7.

9. The pharmaceutical composition of claim 6, wherein the one or more cellulose polymers are selected from the group consisting of: microcrystalline cellulose, croscarmellose sodium, hydroxymethyl cellulose, hydroxyethyl cellulose, hydroxypropyl cellulose, hydroxypropyl methyl cellulose acetate succinate and methyl cellulose.

10. The pharmaceutical composition of claim 9, wherein the lactose is selected from the group consisting of lactose monohydrate and lactose anhydrous.

11. The pharmaceutical composition of claim 10, further comprising colloidal silicon dioxide or magnesium stearate.

12. The pharmaceutical composition of claim 5, wherein the pharmaceutical composition is in a spray-dried dispersion.

13. The pharmaceutical composition of claim 5, wherein the pharmaceutical composition is in tablet form.

14. The pharmaceutical composition of claim 5, comprising about 40-60% of a mesylate salt of the compound of Formula (I).

15. The pharmaceutical composition of claim 5, comprising a dose of about 200 mg of a mesylate salt of the compound of Formula (I).

16. The pharmaceutical composition of claim 15, consisting of the composition according to the table below:

| Ingredients | % weight |
|---|---|
| mesylate salt SDD powder | 50 |
| Lactose Monohydrate (310 NF grade) | 25 |
| Microcrystalline Cellulose (Avicel PH101) | 7 |
| Croscarmellose Sodium (Ac-di-sol) | 8 |
| Colloidal Silicon Dioxide (Cab-o-sil) | 0.5 |
| Magnesium Sterate | 0.5 |
| Croscarmellose Sodium (Ac-di-sol) | 8 |
| Colloidal Silicon Dioxide (Cab-o-sil) | 0.5 |
| Magnesium Sterate | 0.5 |

17. The pharmaceutical composition of claim 15, formulated for oral administration.

18. The pharmaceutical composition of claim 15, wherein the mesylate salt is in crystalline form.

19. The pharmaceutical composition of claim 18, wherein the crystalline mesylate salt is characterized by an X-ray powder diffraction pattern with peaks (2θ±0.3°) at 11.0, 12.5, 13.7, 14.8, 17.6, 22.3, 23.2 26.0, 28.7.

20. The pharmaceutical composition of claim 15, the mesylate salt comprises at least one solid form described in Table 11.
